SEQUENCE LISTING (1 sequence)

SEQ ID NO: 1 - protein, 427 aa, Homo sapiens
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

(19) United States
(12) Patent Application Publication
(10) Pub. No.: US 2010/0061981 A1
(43) Pub. Date: Mar. 11, 2010

(54) p75NTR MEDIATES EPHRIN-A REVERSE SIGNALING

(75) Inventors: Dennis D.M. O'Leary, San Diego, CA (US); Yoo-Shick Lim, San Diego, CA (US); Todd McLaughlin, La Jolla, CA (US)

Correspondence Address: Joseph R. Baker, APC;Gavrilovich, Dodd & Lindsey LLP, 4660 La Jolla Village Drive, Suite 750, San Diego, CA, 92122, US

(73) Assignee: THE SALK INSTITUTE FOR BIOLOGICAL STUDIES, La Jolla, CA (US)

(21) Appl. No.: 12/542,457

(22) Filed: Aug. 17, 2009

Related U.S. Application Data
(60) Provisional application No. 61/089,421, filed on Aug. 15, 2008.

(51) Int. Cl. A61K 39/395; A61K 38/16; A61K 31/7105; A61K 31/7088; G01N 33/53; A61P 35/04

(52) U.S. Cl. 424/130.1; 514/12; 514/44 A; 514/44 R; 435/7.21

(57) ABSTRACT

The disclosure comprises methods and compositions for stimulating axon outgrowth and inhibiting metastatic diseases and disorders.

### CROSS REFERENCE TO RELATED APPLICATIONS

This application claims priority under 35 U.S.C. §119 from Provisional Application Ser. No. 61/089,421, filed Aug. 15, 2008, the disclosure of which is incorporated herein by reference.

### TECHNICAL FIELD

This invention relates methods and compositions useful for modulating metastasis, treating cancer, and modulating neuronal development, cell migration and axon growth.

### BACKGROUND

Cytoskeletal protein and signaling and extracellular matrix interactions provide cues to a cell as it develops, matures and interacts with its environment. Axons, for example, respond to a complex environment of guidance cues as they pathfind, and once within their target, to form an orderly set of connections termed a topographic map.

Similarly, environmental cues and cytoskeletal and second messenger systems within neoplastic cells provide stimuli that promote metastasis and tissue invasion.

### SUMMARY

Unlike most receptor-ligand interactions, the Eph receptor tyrosine kinases and their ligands, the ephrins, have the added complexity that ephrins can act as receptors with Ephs as ligands. This ‘reverse signaling’ has been a conundrum for the ephrin-A subfamily, because they lack an intracellular domain requiring association with transmembrane proteins to transduce their signals. The disclosure shows that the neurotrophin receptor, p75NTR (p75NTR), known for roles in apoptosis and degeneration, complexes with ephrin-As in caveolae in the axon membrane. This ephrin-A-p75NTR complex activates intracellular pathways that involve Fyn, a Src family kinase, and is required for axon repulsion in response to EphAs. Mice lacking p75NTR have defects in axon guidance and mapping in the visual system that reflect the loss of ephrin-A-p75NTR reverse signaling. These discoveries underscore the importance of unique interactions between protein families known for distinct functions in development.

The disclosure provides a method of treating a neurological disease, disorder or injury, comprising: contacting a nerve location with an antagonist agent of p75NTR.

The disclosure also provides a method of treating a neurological disease, disorder or injury, comprising: contacting a nerve location with an agent that inhibits the interaction of p75NTR with an ephrin A.

The disclosure further provides a method of stimulating axonal outgrowth comprising contacting a nerve with an agent that inhibits the interaction of P75NTR with an ephrin A.

The disclosure provides a method of stimulating axonal outgrowth comprising contacting a nerve with an agent that antagonizes p75NTR activity.

In one embodiment, the agent comprises a soluble EphA extracellular domain; an antisense molecule that inhibits expression of p75NTR, Fyn or an ephrin A; an siRNA molecule that inhibits expression of a p75NTR, a Fyn or an ephrin A; an antibody that binds to p75NTR and inhibits the interaction of p75NTR with ephrin A; an antibody binds to ephrin A and inhibits the interaction of ephrin A with p75 NTR; a small molecule inhibitor; a mutant p75NTR lacking a cytoplasmic domain, or an agent that inhibits the binding of endogenous EphA to ephrin A (e.g., a soluble EphA-Fc or an antibody directed against the EphA binding sites on an ephrin A).

The disclosure further provides a method of treating mono or polyneuropathy comprising contacting a subject with an agent that inhibits the interaction of p75NTR with an ephrin A or an antagonist of ephrinA-p75NTR complex activity.

The disclosure also provides a method of treating metastasis comprising contacting a subject with a metastatic disorder or disease with an agent that promotes the interaction of p75NTR and ephrin A or with an agonist of p75-ephrin A complex activity. In one embodiment, the agent comprises a polynucleotide encoding p75NTR; a polynucleotide encoding ephrin A; or an agent the induces phosphorylation of Fyn.

The disclosure also provides a method of screening an agent that is useful for inducing axon outgrowth or cell motility comprising contacting a cell with the agent and measuring (i) the phosphorylation of Fyn or (ii) the interaction of p75NTR and ephrin A, wherein an agent the promotes phosphorylation of Fyn or the interaction of P75NTR and ephrin A is an agent useful for simulating axon outgrowth.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A-E shows retinal expression and Co-localization of p75NTR and Ephrin-As. Cryosections at 20 μm of P2 wild type mouse stained with DAPI to label nuclei (blue). (A and B) Retina immunolabeled with (A) anti-ephrin-A5 (red) and (B) anti-ephrin-A2 (red). Ephrin-A5 and ephrin-A2 are present in the ganglion cell layer (GCL; arrows), retinal ganglion cells (RGCs), RGC axons, and the optic nerve (on). Ephrin-A5 and ephrin-A2 are present in a high to low nasal (N)-temporal (T) gradient. (C) Sagittal section through superior colliculus (SC) labeled with ephrin-A5-Fc affinity probe. EphAs are in a high to low anterior (A) to posterior (P) gradient in superficial layers of SC (arrow; red). (D and E) Retina immunolabeled with anti-p75NTR shown at low (D) and high (E) magnification. p75NTR (red) is present throughout retina, including the GCL, RGC axons (arrows), and optic nerve. (F to F″) Mouse retinal axon in vitro double-labeled with (F) anti-p75NTR and (F′) anti-ephrin-A5. Discrete domains of p75NTR and ephrin-A5 on the cell body (asterisks) and its processes are evident. (F″) Overlap of p75NTR (green) and ephrin-A5 (red) labeling demonstrates their co-localization (yellow; arrows), though clear domains of each are visible (arrowhead). Co-localized domains are in close proximity to domains of p75NTR and ephrin-A5 (inset). Scale bar=50□m in A to D, 15 μm in E, 8 μm in F-F″.

FIG. 2A-B shows ephrin-As and p75NTR are present in the same complex. (A) Retina from wild type and p75NTR null mutant mice immunoprecipitated (IP) with anti-p75NTR antibody (Buster) or anti-ephrin-A2 antibody (R&D systems). Western blots (WB) reveal that p75NTR and ephrin-A2 co-IP. (B) PC12 cells and PC12 cells stably transfected with V5-ephrin-A2 immunoprecipitated with the antibody indicated. Western blots demonstrate that p75NTR (endogenously expressed by PC12 cells) and V5-ephrin-A2 co-IP. (C) Triple immunolabeled 293 cells transfected with V5-ephrin-A2 or cMyc-p75NTR. Cells were incubated with EphA7-Fc and triple-labeled with antibodies against cMyc, V5, and Fc. Both p75NTR (red, arrowhead) and ephrin-A2 (blue) are in a punctate distribution on distinct cells. EphA7-Fc (green, arrow) labels only cells transfected with V5-ephrin-A2. Cells are also stained with DAPI (white; nuclei). Western blots after IPs with the antibodies listed on 293T cells transiently transfected with the construct(s) indicated (+) demonstrate that ephrin-A2 and ephrin-A5 co-IP with p75NTR.

FIG. 3A-C show EphA-induced Fyn phosphorylation in Caveolae requires p75NTR. Stably transfected V5-ephrin-A2, p75NTR and V5-ephrinA2/p75NTR 293 cells treated with Human-Fc (2 μg/ml) or EphA7-Fc (2 μg/ml) for 10 minutes at 37° C. Cells were lysed and fractionated through a sucrose gradient (see Experimental Procedures). The presence of the caveolae (cav) associated protein flotillin-1, detected with an anti-flotillin-1 antibody, and GM1, detected with CTX-HRP in a dot blot, indicates the fractions containing caveolae. Tyrosine phosphorylation (p-Tyr; 4G10 antibody) in the caveolae fractions is unchanged when challenged with EphA7-Fc compared to Fc in both the (A) ephrin-A2 cell line and the (B) p75NTR cell line. (C) In contrast, the ephrin-A2/p75NTR cell line has a higher level of p-Tyr in caveolae fractions (arrowheads) when treated with EphA7-Fc compared to Fc alone (arrows). The largest increase in p-Tyr (arrowheads) is coincident with the location of Fyn on the re-probed blot (hollow arrowheads).

FIG. 4A-D shows retinal axons require p75NTR for EphA7 repulsion. In vitro protein stripe assays demonstrating that wild type RGC axons preferentially avoid stripes containing EphA7 but p75NTR^{-/-} RGC axons do not. (A and B) Axons (green) extending on a control substrate of alternating stripes of human-Fc and human-Fc (Fc). Axons do not show a growth preference for one stripe over the other whether they extend from (A) a p75NTR^{+/+} mouse retinal explant or from (B) a p75NTR^{-/-} retinal explant. (C and D) Axons extending on a substrate of alternating stripes of human-Fc and EphA7-Fc (red; A7). (C) Axons from a p75NTR^{+/+} retinal explant preferentially extend on the human-Fc stripes and avoid the EphA7 stripes. (D) In contrast, axons from a p75NTR^{-/-} retinal explant do not avoid stripes containing EphA7. (E and F) Axons extending on a substrate of alternating stripes of human-Fc and ephrin-A5-Fc (red; A5). Axons show a strong preference for the Fc containing stripes and avoid the ephrin-A5 containing stripes whether they extend from (E) a p75NTR^{+/+} retinal explant or from (F) a p75NTR^{-/-} retinal explant. Scale bar=200 μm.

FIG. 5A-D shows statistical analysis of stripe assay results. (A) Average growth preference scores (error bars=s.e.m.) for retinal axons in the stripe assay (see Experimental Procedures). A score of four is an essentially complete choice for one stripe, a score of zero is no discernible choice for either stripe. Retinal axons do not show a preference on control human-Fc vs human-Fc (Fc) substrates. Retinal axons from p75NTR^{+/+} explants show significant avoidance of EphA7 compared to retinal axons from p75NTR^{-/-} explants. In contrast, axons extending from p75NTR^{+/+} or p75NTR^{-/-} explants avoid ephrin-A5 (A5) to a similar extent. (B) The coefficient of choice for p75NTR^{+/+} and p75NTR^{-/-} axons extending on control Fc vs Fc substrates or EphA7-Fc vs Fc substrates is shown. Pixels representing axons present in each stripe were quantified and the coefficient calculated as the number of pixels on the Fc stripe minus pixels on the second stripe (Fc or EphA7), divided by total pixels (see Experimental Procedures). A coefficient of one is an absolute choice for the control stripe and a coefficient of zero indicates no preference. p75NTR^{+/+} axons preferentially avoid EphA7 stripes, whereas p75NTR^{-/-} axons do not show a significant preference for Fc stripes compared to EphA7 stripes. (C and D) Protein stripe assays analyzed with a simplified Sholl intersection analysis. (C) Schematic demonstrates the analysis method (see Experimental Procedures). All intersections (arrowheads) between axons and lines at defined distances from the explant edge were counted blind to genotype, stripe content, and stripe position. (D) Coefficients of choice for intersection points determined by the modified Sholl analysis (intersections on the Fc stripe minus intersections on the second stripe (Fc or EphA7 or ephrin-A5), divided by total intersections). On Fc vs Fc substrates there is no significant choice for either stripe. In contrast, p75NTR^{+/+} axons extending on Fc vs EphA7 substrates intersect the Sholl lines significantly more often on Fc stripes than on EphA7 stripes. However, in p75NTR^{-/-} axons, this preference is greatly reduced and the number of intersection points on Fc and EphA7 stripes is not significantly different. For both p75NTR^{+/+} and p75NTR^{-/-} axons extending on Fc vs ephrin-A5 substrates, significantly more intersection points occur on Fc stripes compared to ephrin-A5 stripes. N values in panel A apply to panels B and D. n.s., not significant; *, p<0.02; **, p<0.001.

FIG. 6A-F shows aberrant retinocollicular mapping in p75NTR knockout mice. (A) Dorsal view of the superior colliculus (SC) of a P8 wild type mouse after focal injection of DiI in nasal (N) retina reveals a dense termination zone (TZ) in posterior (P, dotted line is posterior SC border) SC. No interstitial branches are evident in the SC outside of the TZ at this age in p75NTR^{+/+} mice. Arrowheads mark the anterior (A) border. (B) SC of a P8 p75NTR^{-/-} mouse injected with DiI in nasal retina (injection is similar in size and location to that in panel A), reveals a dense TZ in posterior SC, but shifted anteriorly (sTZ) in comparison to wild type. Multiple branches (arrows) and rudimentary arbors (black arrowhead) are evident throughout the SC, anterior to the TZ. (C) Schematic describing the expression of cre-recombinase in nasal and temporal retinal ganglion cells (RGCs; red) in a cre mice. The α-cre line in combination with the ROSA-GAP43-eGFP (R-eGFP) line results in a stereotypic pattern of eGFP labeled RGC axons (green) in three distinct domains in the SC, corresponding to the eGFP-labeled projection from temporal (T^{d}) and nasal (N^{d}) retina, and the unlabeled central domain (C^{d}). (D) Dorsal view of the SC of a p75NTR^{+/+}; α-cre; R-eGFP mouse illustrating the stereotypic pattern of R-eGFP in wild type mice. Bracket denotes the anterior-posterior extent of the N^{d}. (E and F) RGC projections in p75NTR^{-/-}; α-cre; R-eGFP mice show an anterior shift in nasal RGC axon mapping. (E) Bracket denotes an extended, anteriorly shifted N^{d}. (F) In some p75NTR^{-/-} cases the eGFP is discontinuous and has gaps (arrow) indicating a disorganized projection. These gaps are not observed in p75NTR^{+/+}; α-cre; R-eGFP mice. L, lateral; M, medial. Scale bar=400 μm.

FIG. 7A-C shows conditional allele of p75NTR is excised with cre recombinase. (A and A') Cryosection through a P2 p75NTR^{+/+}; α-cre; R-eGFP mouse. The nasal (N) and temporal (T) embodiments of the retina, including the ganglion cell layer (GCL), are labeled with eGFP, mimicking cre expression. The distribution of p75NTR is unaffected in p75NTR^{+/+}; α-cre; R-eGFP mice. (B and B″) However, in p75NTR fl/fl; α-cre; R-eGFP mice, p75NTR protein is not detectable at P2 in nasal and temporal retina, but unchanged in central retina. The eGFP label in panel B indicates the presence of cre-recombinase and, thus, the cells in which p75NTR has been excised. The arrows are in the same position and denote the border of eGFP expression. (C and C′) Cryosection from the retina of a p75NTR fl/fl; α-cre; R-eGFP mouse labeled for the RGC marker Brn3.2 at P2. The proportion of RGCs in central retina, where p75NTR expression is unaltered, is identical to that in nasal and temporal retina, where p75NTR is absent. Arrowheads denote the edges of cre expression. Scale bar=40 μm.

FIG. 8A-F shows aberrant retinocollicular mapping in p75NTR conditional mice. (A) Dorsal view of the superior colliculus (SC) of p75NTR fl/fl; cre-negative mouse at P8 after focal injection of DiI in nasal retina reveals a dense termination zone (TZ) in posterior (P) SC (dotted line is posterior SC border; arrowheads mark the anterior (A) SC border). (B and C) SCs of p75NTR fl/fl; α-cre mice at P8 after focal injections of DiI in nasal retina, similar in size and location to that in panel A. (B) In every p75NTR fl/fl; α-cre case the TZ is shifted (sTZ) anteriorly, compared to its expected position. (C) In a subset of p75NTR fl/fl; α-cre mice focal DiI injection reveals two TZs in posterior SC. The arrow points to the appropriate location of the TZ, with a sTZ in an anterior position. (D) A p75NTR^{+/+}; α-cre; R-eGFP case illustrates the stereotypic pattern of the eGFP labeled temporal and nasal RGC axon projection domains (T^{d} and N^{d} respectively). The projection domain from central retina (C^{d}) is unlabeled. (E and F) In p75NTR fl/fl; α-cre; R-eGFP mice, the N^{d} of is significantly expanded anteriorly and the C^{d} is significantly reduced. In many p75NTR fl/fl; α-cre; R-eGFP cases the eGFP is discontinuous and has a mottled appearance, suggesting a disorganized map (arrow in E). L, lateral; M, medial. Scale bar=400 μm.

FIG. 9A-D shows quantification and summary of retinocollicular shifts in p75NTR mutant mice. (A) Average position of the center of the DiI-labeled termination zones (TZs) from the posterior pole of the superior colliculus (SC) in percent of the anterior-posterior (AP) extent of the SC. There is a significant anterior shift in TZ position for p75NTR^{-/-} and p75NTR fl/fl; α-cre mice compared to controls. The positions of retinal injection sites between genotypes are not statistically distinct. (B) Box plots illustrating the distributions of TZ locations. The top and bottom edges of each box are the 25^{th} and 75^{th} percentile, respectively. The horizontal line within each box is the median value. The vertical ‘whiskers’ extend above and below each box to the most divergent point within three times the interquartile value. Filled circles are outliers. The distribution of TZ positions for p75NTR^{+/+} and p75NTR fl/fl; cre negative are not different. However, p75NTR^{-/-} and p75NTR fl/fl; α-cre mice have TZ distributions significantly shifted anteriorly. Mann-Whitney U-test p-values for the pairs indicated: n.s., not significant; *, p<0.01; **, p<0.001. (C) Borders of R-eGFP labeling superimposed on a dorsal view of the SC for the p75NTR^{+/+}; α-cre; R-eGFP cases in FIGS. 6D and 8D (red). Two representative p75NTR^{-/-}; α-cre; R-eGFP cases (blue; from FIGS. 6E and 6F) illustrate the anterior shift of the nasal domain (N^{d}). The p75NTR fl/fl; α-cre; R-eGFP case shown in FIG. 8E is illustrated in green. Note the large anterior shift of the N^{d} and a reduced central domain (C^{d}). (D) Average projection domain areas superimposed on a dorsal view of the SC. The lines indicate the average AP positions for borders of eGFP labeling for p75NTR^{+/+}; α-cre; R-eGFP mice (red) and p75NTR fl/fl; α-cre; R-eGFP mice (green). The values for p75NTR^{+/+} cases (red) indicate the area of the SC each domain occupies, whereas the values for p75NTR fl/fl; α-cre cases (green) represent the percentage change from wild type. There is a significant expansion and anterior shift of the N^{d} (p<0.01) and a concomitant significant decrease in the C^{d} in p75NTR fl/fl; α-cre mice compared to control mice (p<0.01).

FIG. 10A-B shows a summary of the retinocollicular mapping defects in p75NTR mutant mice. (A) In wild type mice, ephrin-As expressed in the retina (green gradient) and along retinal ganglion cell (RGC) axons interact with EphAs (blue gradient) in the superior colliculus (SC). In addition, p75NTR is expressed in the retina (orange) and along RGC axons and acts as an ephrin-A signaling partner. Therefore, p75NTR complexes with ephrin-As along RGC axons and, upon binding EphAs in the SC, transduces a repellent ephrin-A reverse signal (red gradient) that parallels the anterior (A)-posterior (P) EphA gradient in the SC. Thus, nasal (N) RGC axons, expressing high levels of ephrin-As, form a termination zone (TZ) in posterior SC, which expresses low levels of EphAs. (B) In p75NTR mutant mice an ephrin-A signaling partner is lacking from the retina. Thus, the repellent ephrin-A reverse signal is reduced (diminished red gradient) allowing nasal RGC axons to form anteriorly shifted TZs (sTZ). The expression patterns of ephrin-As and EphAs are unchanged in p75NTR mutant mice. Therefore, the formation of sTZs in areas of high EphA expression is due to the loss of the ephrin-A signaling partner, p75NTR, and the concomitant reduction in repellent ephrin-A reverse signal.

### DETAILED DESCRIPTION

As used herein and in the appended claims, the singular forms “a,” “and,” and “the” include plural referents unless the context clearly dictates otherwise. Thus, for example, reference to “a probe” includes a plurality of such cells and reference to “the primer” includes reference to one or more primers and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure.

Diverse protein families affect axon pathfinding and mapping, including but not limited to, semaphorins, Wnts, neurotrophins, ephrins, and their cognate receptors (Tessier-Lavigne and Goodman, 1996; Huber et al., 2003; McLaughlin and O'Leary, 2005; Flanagan 2006). Signaling from diverse families of guidance molecules must converge to provide coherent guidance information. Though axon guidance systems eventually link to the cytoskeleton, distinct families of guidance molecules and receptors interact at multiple points in their signaling pathways, from ligand binding to intramembrane interactions to cytoskeletal alterations (Grunwald and Klein, 2002; Kullander and Klein, 2002; Murai and Pasquale, 2003).

The Eph tyrosine kinase subfamily is a large subfamily of transmembrane receptor tyrosine kinases. A unified nomenclature has been developed in which the Eph receptors are divided into two groups on the basis of sequence homologies. Ephs and ephrins are each separated into A and B subclasses that exhibit promiscuous receptor-ligand binding and activation within each subclass, but little between subclasses (Gale et al., 1996). All Eph receptors, as well as ephrin-Bs, are transmembrane proteins, whereas ephrin-As are GPI-linked to the cell membrane. In addition, EphB-ephrin-B binding can result in bidirectional signaling, characterized by not only “forward” signaling into cells that express EphBs, but also “reverse” signaling into ephrin-B-expressing cells. Reverse signaling by ephrin-Bs is accomplished by association of the intracellular domain of ephrin-Bs with intracellular kinases and phosphatases (Cowan and Henkemeyer, 2002; Kullander and Klein, 2002). EphAs and ephrin-As can also transduce signals bidirectionally, indicating that ephrin-As reverse signal even though they lack an intracellular domain (Davy et al., 1999; Davy and Robbins, 2000; Huai and Drescher, 2001). Reverse signaling by ephrin-As has been implicated in the pathfinding of vomeronasal (Knoll et al., 2001) and spinal motor axons (Marquardt et al., 2005), and the topographic mapping of the axons of olfactory neurons (Cutforth et al., 2003) and retinal ganglion cells (RGCs; Rashid et al., 2005).

Because ephrin-As are anchored to the cell membrane by a GPI linkage and lack an intracellular domain, to reverse signal they must associate with transmembrane proteins capable of activating intracellular signaling pathways. Examples of such associations between GPI-anchored proteins and transmembrane signaling partners in neurons include the transmembrane protein CASPR and the GPI-linked cell adhesion molecule contactin (Peles et al., 1997) and binding of GDNF to the receptor complex formed by the GPI-anchored receptor GFRα1 and the transmembrane protein c-Ret (Jing et al., 1996, Trupp et al., 1998). However, a transmembrane signaling partner for ephrin-As has not been reported.

Axon repulsion is required for guidance and topographic mapping in many neural systems and is mediated by reverse signaling by ephrin-As. Ephrin-A5 and p75NTR co-immunoprecipitate, indicating that they are present in the same complex. In vitro protein stripe assays demonstrate that wild type retinal axons avoid EphA7, but retinal axons from mice deficient for p75NTR do not. Thus, p75NTR is required for EphA mediated axon repulsion by acting as a co-receptor required for ephrin-A reverse signaling. Mice lacking p75NTR or with floxed p75NTR alleles selectively deleted from retina have aberrant retinotopic mapping in the superior colliculus (SC). Using the fluorescent axon tracer DiI to label small numbers of retinal ganglion cells axons from the same location in retina, in p75NTR mutant mice, termination zones (TZ) that are located in aberrantly anterior positions in the SC. In some cases a double TZ is observed, with one TZ in the appropriate topographic location and a second TZ located in an aberrant anterior position. These results are consistent with diminished repellent activity mediated by ephrin-A reverse signaling in response to the high-to-low anterior-to-posterior gradient of EphAs in the SC. In addition the entire projection to the SC from the nasal and temporal portions of the retina was analyzed through the use of the α-cre transgenic line and a cre-recombinase activated eGFP marker, either on a wild type background or crossed to the p75 knockout mice or mice with floxed alleles of p75NTR. In p75NTR deficient mice the entire nasal retinal projection domain in the SC is significantly shifted anteriorly. This result is consistent with diminished repellent activity mediated by ephrin-A reverse signaling. This demonstrates that p75NTR is an ephrin-A co-receptor in retinal axons, mediates their repulsion due to ephrin-A reverse signaling, and is required for appropriate retinocollicular mapping.

The disclosure demonstrates that ephrin-As and p75NTR associate in caveolae along RGC axons, and that p75NTR transduces the ephrin-A reverse signal that repels RGC axons and is required for proper AP mapping in the SC. The disclosure demonstrates that ephrin-As and p75NTR form a complex that results in ephrin-A's reverse signaling and apoptosis. The disclosure further demonstrates that p75NTR is required for EphA7 to induce a significant increase in the phosphorylation of Fyn in caveolae, indicating that the Fyn signaling pathway associated with ephrin-A reverse signaling is p75NTR dependent. In vitro guidance assays show that p75NTR is required for retinal axons to be repelled by EphAs. The disclosure further demonstrates that p75NTR acts as a signaling partner with ephrin-As to mediate the repellent effect of ephrin-A reverse signaling on RGC axons upon binding EphAs, and that this signaling is required for appropriate retinotopic mapping.

It is important to note that evidence suggest that EphA concentrations can have differing effects on certain cell types. For example, low concentrations may promote one activity while higher concentrations promote another. Accordingly, the function of the p75NTR-ephrin A complex can be inhibitory/repulsive or conversely stimulatory/attractant—the function of ephrins is context dependent.

The interaction (or complexing) of ephrin-As with p75NTR results in a reverse signaling inhibiting cytoskeltal development and thus axonal development. A p75NTR-ephrin A complex induces a kinase cascade ultimately leading Fyn phosphorylation leading to cytoskeletal changes and reducing axonal development. As will also be understood cytoskeletal changes also play a role in metastatic diseases and disorders whereby promoting cancer cell tissue invasion.

Functional evidence for the p75-ephrin-A signaling complex is provided by the in vitro axon guidance assays described below and show that p75 is required for the repulsion of retinal axons by EphA. In contrast, p75 is not required for the repulsion of retinal axons by ephrin-A. These data indicate that p75 is selectively required for the repellent guidance activity mediated by ephrin-A reverse signaling, but is not required for the repellent guidance activity mediated by EphA forward signaling.

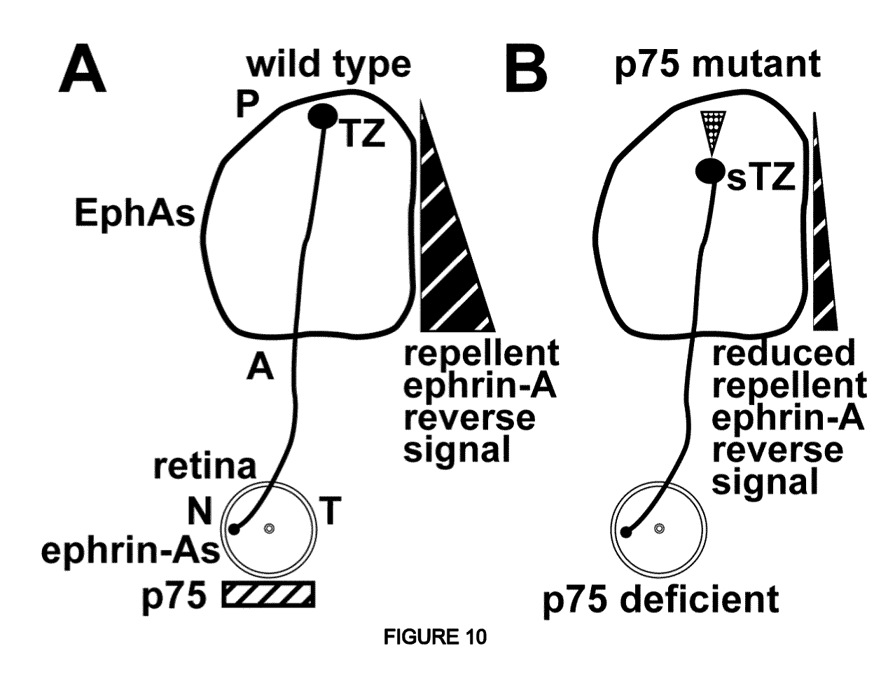

The disclosure demonstrates that p75 is required for appropriate topographic mapping of RGC axons in the SC, by analyzing mice constitutively null for p75 or in which floxed alleles of p75 are selectively deleted from retina. In both p75 mutants, essentially all RGC axons aberrantly terminate anterior to their topographically appropriate position in the SC. This anterior shift in the terminations of p75 deficient RGC axons is the predicted outcome if the repellent activity of ephrin-A reverse signaling along the AP axis of the SC is diminished and p75 mediates this signaling (FIGS. 10A and 10B). These data from each set of experiments in the study support the conclusion that p75 complexes with ephrin-A in RGC axon membranes, and that p75 is required for the transduction of a repellent signal to RGC axons when axonally expressed ephrin-A binds EphA.

For example, p75^{-/-} mice have normal retinal morphology and numbers of RGCs and no obvious defects in the retina or SC in either p75^{-/-} mice or p75 fl/fl; α-cre mice, including the expression of ephrin-As, EphAs, and RGC markers. The α-cre mice used to delete p75 from retina in the conditional p75 knockout mice have the important feature that cre-recombinase is not expressed in the SC or anywhere in the visual pathway outside of the retina. In addition, the early phases of map development appear similar in p75 mutants compared to wild type mice. These observations, the similarity in mapping defects in the two distinct p75 mutant lines, and the consistency of the mapping defects in p75 mutant mice with results from the in vitro axon guidance assays, show that the aberrant phenotypes are due to the lack of p75 in RGC axons, and are not due to secondary effects.

The aberrant mapping observed in the p75 mutant mice is very consistent and is the predicted phenotype for a diminished action of ephrin-A reverse signaling. The data demonstrate that the mapping defect is characterized by the formation of a relatively normal appearing TZ at an aberrant anterior position in the SC indicates that p75 deficient RGC axons are affected in a uniform manner, with essentially all RGC axons exhibiting a diminished response to ephrin-A repulsion. It is possible that the anterior shift in terminations in p75 mutant mice is effected by the action of another signaling partner for ephrin-A partially redundant with p75, for example TROY, a transmembrane receptor that shares features with p75. Because essentially all p75 deficient RGC axons are affected in a uniform manner, the competitive balance between them would be retained, and would act to limit the magnitude of the anterior shift of their terminations. For example, competitive interactions limit the aberrant posterior shift in the terminations of RGC axons genetically engineered to express higher levels of EphA, and therefore experience higher levels of repellent EphA forward signaling (Brown et al., 2000).

The classic function of p75 is as an NTR. BDNF or other neurotrophins (e.g., either acting through P75 or complexed with Trk receptors), a neurotrophin ligand for the high affinity NTR, TrkB, also binds p75, and has a general role as a growth promoter of RGC axon arbors in the retinotectal projection in Xenopus (Cohen-Cory and Fraser, 1995; Alsina et al., 2001). The disclosure demonstrates that p75 complexing with ephrin-A mediates the repellent effect of ephrin-A reverse signaling, rather than p75 mediating growth promoting effects of BDNF. For example, in the stripe assay wild type retinal axons are strongly repelled by EphAs, but p75 null retinal axons are not affected, though in vitro, explants from wild type and p75 null mice extend the same number of axons and their average distance of extension is the same. Further, in vivo, p75 deficient RGC axons exhibit their normal, initially exuberant growth across the SC. Thus, in the absence of p75, axon repulsion due to ephrin-A reverse signaling is lost, but general features of axon growth are normal.

In addition, Fyn, which is a prominent component of the p75-ephrin-A signaling pathway, is also an important contributor to the signaling pathways of other guidance molecules, including netrin/DCC and sema3A (Sasaki et al., 2002; Liu et al., 2004; Meriane et al., 2004). Thus, p75 acts broadly as a partner for ephrin-A reverse signaling and potentially other families of axon guidance molecules and their signaling pathways suggesting that both p75 and Fyn are involved in integrating multiple signaling pathways to provide coherent guidance information.

Accordingly, the disclosure provides methods and compositions useful in modulating axonal development and cellular apoptosis. For example, by modulating (i.e., stimulating or inhibiting) the interaction of p75NTR and ephrin-A one can affect axon guidance and growth or inhibit metastasis.

The term “Eph receptor” refers to a tyrosine kinase receptor which belongs to the Eph family of receptors. The Eph family comprises at least fourteen structurally related transmembrane receptor tyrosine kinases, each having a extracellular region comprising a series of modules; a putative immunoglobulin (Ig) domain at the amino terminus, followed by a cysteine-rich region and two fibronectin type III repeats near the single membrane-spanning segment, a cytoplasmic region comprising a highly conserved tyrosine kinase domain flanked by a juxtamembrane region and a carboxyl-terminal tail, which are less conserved.

Eph receptors of the EphA group, designated “EphA receptors” herein, interact with glycosylphosphatidylinositol (GPI)-linked ligands (of the Ephrin-A subclass). Specific EphA receptors include: EphA1 (also called Eph and Esk); EphA2 (also called Eck, mEck, Myk2, Sek2); EphA3 (also termed Hek, Mek4, Tyro4 and Cek4); EphA4 (also known as Hek8, Sek1, Tyro1, and Cek8); EphA5 (also called Hek7, Bsk, Ehk1, Rek7 and Cek7); EphA6 (also called mEhk2 and Ehk2); EphA7 (otherwise named Hek 11, Mdk1, Ebk, Ehk3); and Eph8 (also termed Eek and mEek) and naturally occurring variants thereof.

An Eph-ligand generally refers to a polypeptide which binds to and, optionally, activates (e.g. stimulates tyrosine phosphorylation of) an Eph receptor.

The Eph ligand may be a GPI-linked Eph ligand, i.e. comprising a glycosylphosphatidylinositol or GPI anchor. GPI-linked Eph ligands include, for example, Ephrin-A1 (Lerk1 and B61); Ephrin-A2 (Elf1 and Cek7-L); Ephrin-A3 (Lerk3 and Ehk1-L); Ephrin-A4 (Lerk4); and Ephrin-A5 (Lerk7, All and Rags).

The term “soluble Eph receptor” or “soluble Eph ligand” herein refers to a form of the Eph receptor or Eph ligand which is essentially free of a membrane anchoring region of the native molecule or a form which has an inactivated membrane anchoring region. By “membrane anchoring region” is meant a transmembrane domain (and optionally a cytoplasmic domain) of an Eph receptor or Eph ligand, or a GPI anchor of an Eph ligand.

A soluble Eph receptor useful in the methods and compositions of the disclosure modulate p75NTR-ephrin-A interactions and can take the form of multimer, dimer or trimer. Soluble domains of Ephrin-A or EphA may be fused to molecules such as peptide linkers or immunoglobulins for purposes increasing the valency of polypeptide binding sites. For example, fragments of a soluble EphA or soluble Ephrin-A may be fused directly or through linker sequences to the Fc portion of an immunoglobulin. For a bivalent form of the polypeptide, such a fusion comprises an Fc portion of an IgG molecule. Other immunoglobulin isotypes may also be used to generate such fusions. For example, a polypeptide-IgM fusion would generate a decavalent form of the polypeptide of the disclosure. The term “Fc polypeptide” as used herein includes native and mutein forms of polypeptides made up of the Fc region of an antibody comprising any or all of the CH domains of the Fc region. Truncated forms of such polypeptides containing the hinge region that promotes dimerization are also included.

In one embodiment, an Fc polypeptides comprise an Fc polypeptide derived from a human IgG1 antibody. Preparation of Fusion Polypeptides Comprising Certain heterologous polypeptides fused to various portions of antibody-derived polypeptides (including the Fc domain) has been described (see, e.g., by Ashkenazi et al. PNAS USA 88:10535, 1991; Byrn et al. Nature 344:677, 1990; and Hollenbaugh and Aruffo, “Construction of Immunoglobulin Fusion Polypeptides”, in Current Protocols in Immunology, Suppl. 4, pages 10.19.1-10.19.11, 1992). In one embodiment, a dimer comprising two fusion polypeptides created by fusing two soluble domain to an Fc polypeptide derived from an antibody. A gene fusion encoding the polypeptide/Fc fusion polypeptide is inserted into an appropriate expression vector. Polypeptide/Fc fusion polypeptides are expressed in host cells transformed with the recombinant expression vector, and allowed to assemble much like antibody molecules, whereupon interchain disulfide bonds form between the Fc moieties to yield divalent molecules. A suitable Fc polypeptide, described in PCT application WO 93/10151, is a single chain polypeptide extending from the N-terminal hinge region to the native C-terminus of the Fc region of a human IgG1 antibody. Another useful Fc polypeptide is the Fc mutein described in U.S. Pat. No. 5,457,035 and in Baum et al., (EMBO J. 13:3992-4001, 1994). The amino acid sequence of this mutein is identical to that of the native Fc sequence presented in WO 93/10151, except that amino acid 19 has been changed from Leu to Ala, amino acid 20 has been changed from Leu to Glu, and amino acid 22 has been changed from Gly to Ala. The mutein exhibits reduced affinity for Fc receptors. The above-described fusion polypeptides comprising Fc moieties (and oligomers formed therefrom) offer the advantage of facile purification by affinity chromatography over Polypeptide A or Polypeptide G columns. In other embodiments, the polypeptides of the invention can be substituted for the variable portion of an antibody heavy or light chain. If fusion polypeptides are made with both heavy and light chains of an antibody, it is possible to form an oligomer with as many as four soluble EphA extracellular regions. Examples of soluble EphA-receptor/Fc fusion polypeptides.

Alternatively, the oligomer is a fusion polypeptide comprising multiple soluble EphA receptor polypeptides, with or without peptide linkers (spacer peptides). Among the suitable peptide linkers are those described in U.S. Pat. Nos. 4,751,180 and 4,935,233. In some embodiments, a linker moiety separates the soluble EphA polypeptide domain and the second polypeptide domain in a fusion polypeptide. Such linkers are operatively linked to the C- and the N-terminal amino acids, respectively, of the two polypeptides. Typically a linker will be a peptide linker moiety. The length of the linker moiety is chosen to optimize the biological activity of the soluble EphA and can be determined empirically without undue experimentation. The linker moiety should be long enough and flexible enough to allow a soluble EphA moiety to freely interact with a substrate or ligand.

Another method for preparing the oligomers of the disclosure involves use of a leucine zipper. Leucine zipper domains are peptides that promote oligomerization of the polypeptides in which they are found (Landschulz et al., Science 240:1759, 1988), and have since been found in a variety of different polypeptides. Among the known leucine zippers are naturally occurring peptides and derivatives thereof that dimerize or trimerize. The zipper domain or oligomer-forming domain comprises a repetitive heptad repeat, often with four or five leucine residues interspersed with other amino acids. Use of leucine zippers and preparation of oligomers using leucine zippers are known in the art.

The term “antagonist” when used herein refers to a molecule which is capable of inhibiting one or more of the biological activities of a target molecule, such as an Eph receptor. Antagonists may act by interfering with the binding of a receptor to a ligand and vice versa, and/or by interfering with receptor or ligand activation (e.g. tyrosine kinase activation) or signal transduction after ligand binding to a cellular receptor. The antagonist can inhibit the interaction or complexing of Ephrin A-p75NTR or the phosphorylation of Fyn caused by such EphrinA-p75NTR complex. The antagonist may completely block interactions or may substantially reduce such interactions. Thus, included within the scope of the disclosure are antagonists (e.g. neutralizing antibodies) that bind to Eph receptor, Eph ligand or a complex of an Eph receptor and Eph ligand; that bind to p75NTR and inhibit the interaction of p75NTR with Ephrin A; amino acid sequence variants or derivatives of an Eph receptor or Eph ligand which antagonize the interaction between an Eph receptor and Eph ligand or an ephrin A interaction with p75NTR; soluble Eph receptor or soluble Eph ligand, optionally fused to a heterologous molecule such as an immunoglobulin region (e.g. an immunoadhesin); a complex comprising an Eph receptor in association with Eph ligand; synthetic or native sequence peptides which bind to Eph receptor or Eph ligand; small molecule antagonists; and nucleic acid antagonists (e.g. antisense).

Such EphA soluble domains and multimers can act as antagonists of the p75NTR-Ephrin-A complex. In contrast, soluble Ephrin-A domains can act as agonists by promoting the activity of p75NTR.

For example, one approach to block p75NTR-ephrin A complex function includes contacting a site, cell or subject with an agent that prevents the binding of endogenous EphA to ephrin A, for example a soluble EphA-Fc or an antibody directed against the EphA binging sites on ephrin As. Another method can include the use of a soluble EphA receptor that binds to endogenous EphA thereby sequestering the EphA and preventing the endogenous EphA from acting upon endogenous EphA receptors.

An “agonist” herein is a molecule which is capable of activating one or more of the biological activities of the p75NTR-EphrinA target complex. Agonists may, for example, act by activating a target molecule and/or mediating signal transduction. Included within the scope of the disclosure are Eph receptor or Eph ligand themselves; agonists (e.g. agonist antibodies) that bind to an EphA, or form a stimulatory complex with an EphA or p75NTR; amino acid sequence variants or derivatives of a p75NTR or Eph ligand; synthetic or native sequence peptides which bind to and activate Eph receptor or Eph ligand; small molecule agonists; and a gene encoding Eph receptor or Eph ligand (i.e. for gene therapy).

“Treatment” refers to both therapeutic treatment and prophylactic or preventative measures. Those in need of treatment include those already with the disorder as well as those in which the disorder is to be prevented.

“Mammal” for purposes of treatment refers to any animal classified as a mammal, including humans, domestic and farm animals, and zoo, sports, or pet animals, such as dogs, horses, cats, cows, etc. Preferably, the mammal is human. The human to be treated herein includes an embryo or fetus (i.e. wherein the mammal is treated in the uterus), infant, child, pubescent adult or adult.

“Effective amount” or “therapeutically effective amount” of the agonist or antagonist is an amount that is effective either to prevent, lessen the worsening of, alleviate, or cure the treated condition.

A “therapeutically effective amount”, in reference to the treatment of cancer refers to an amount capable of invoking one or more of the following effects: (1) inhibition, to some extent, of tumor growth, including, slowing down and complete growth arrest; (2) reduction in the number of tumor cells; (3) reduction in tumor size; (4) inhibition (i.e., reduction, slowing down or complete stopping) of tumor cell infiltration into peripheral organs; (5) inhibition (i.e., reduction, slowing down or complete stopping) of metastasis; (6) enhancement of anti-tumor immune response, which may, but does not have to, result in the regression or rejection of the tumor; and/or (7) relief, to some extent, of one or more symptoms associated with the disorder.

The term “inhibiting angiogenesis” refers to the act of substantially preventing or reducing the development of blood vessels in a treated mammal.

The expressions “stimulating angiogenesis” or “promoting angiogenesis” refer to the act of substantially increasing the development of blood vessels in a treated mammal.

The term promote neurotropic activity refers to the ability of an inhibitor or antagonist of an ephrin-p75NTR complex to inhibits the reverse signaling of ephrin-p75NTR. A p75NTR-ephrinA antagonist effect can include promoting neurotrophic activity, axon development or growth.

The term inhibits metastasis or ephrin-p75NTR agonist activity refers the ability of an agent that promotes or stimulates the formation or activity of an ephrin-p75NTR complex.

“Diseases or disorders characterized by undesirable or excessive vascularization” include, by way of example, tumors, and especially solid malignant tumors, rheumatoid arthritis, psoriasis, atherosclerosis, diabetic and other retinopathies, retrolental fibroplasia, age-related macular degeneration, neovascular glaucoma, hemangiomas, thyroid hyperplasias (including Grave's disease), corneal and other tissue transplantation, and chronic inflammation.

The disclosure provides methods of influencing central nervous system cells to produce progeny and/or stimulate axonal development and directional growth. Such methods and compositions are useful to replace damaged or missing neurons or to stimulate neuronal development in areas having a neuronal disease, disorder or injury. The methods include exposing a subject suffering from a neurological disease or disorder or injury, to an agent that is an inhibitor of the formation of an ephrin-p75 complex of an antagonist of an ephrin-p75 biological activity. The agent is used in a suitable formulation through a suitable route of administration. A “neurological disease or disorder” is a disease or disorder which results in the disturbance in the structure or function of the central nervous system resulting from developmental abnormality, disease, injury or toxin. Examples of neurological diseases or disorders include neurodegenerative disorders (e.g. associated with Parkinson's disease, Alzheimer's disease, Huntington's disease, Shy-Drager Syndrome, Progressive Supranuclear Palsy, Lewy Body Disease or Amyotrophic Lateral Sclerosis); ischemic disorders (e.g. cerebral or spinal cord infarction and ischemia, stroke); traumas (e.g. caused by physical injury or surgery, and compression injuries; affective disorders (e.g. stress, depression and post-traumatic depression); neuropsychiatric disorders (e.g. schizophrenia, multiple sclerosis or epilepsy); and learning and memory disorders. This disclosure provides a method of treating a neurological disease or disorder comprising administering an inhibitor or antagonist of the ephrin-p75 complex or an inhibitor or antagonist of the reverse signaling induced by an ephrin-p75 complex to a mammal. The term “mammal” refers to any mammal classified as a mammal, including humans, cows, horses, dogs, sheep and cats. In one embodiment, the mammal is a human.

A pharmaceutical composition useful as a therapeutic agent for the treatment of central nervous system disorders is provided. For example, the composition includes an agent of the disclosure, which can be administered alone or in combination with the systemic or local co-administration of one or more additional agents. Such agents include preservatives, ventricle wall permeability increasing factors, stem cell mitogens, survival factors, glial lineage preventing agents, anti-apoptotic agents, anti-stress medications, neuroprotectants, and anti-pyrogenics. The pharmaceutical composition preferentially treats CNS diseases by stimulating cells (e.g., ependymal cells and subventricular zone cells) to proliferate, migrate and differentiate into the desired neural phenotype, targeting loci where cells are damaged or missing.

A method for treating a subject suffering from a CNS disease or disorder is also provided. This method comprises administering to the subject an effective amount of a pharmaceutical composition containing an inhibitory or antagonist (1) alone in a dosage range of 0.5 ng/kg/day to 500 ng/kg/day, (2) in a combination with a ventricle wall permeability increasing factor, or (3) in combination with a locally or systemically co-administered agent.

In another embodiment, the disclosure provides a method of inhibiting a metastatic cell proliferative disease or disorder. The method comprises administering an agent the simulates or is an agonist of ephrin-p75 complexes. In this embodiment, by “promoting” the biological activity associated with ephrin-p75 complexes (e.g., reverse signaling, decreased cytoskeletal development) the ability of metastatic cells to infiltrate or invade a tissue is reduced or inhibited.

Therapeutic formulations of the agonist or antagonist are prepared for storage by mixing the agonist or antagonist having the desired degree of purity with optional physiologically acceptable carriers, excipients or stabilizers [Remington's Pharmaceutical Sciences 16th edition, Osol, A. Ed. (1980)], in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONICS or polyethylene glycol (PEG).

The agonists or antagonists may also be formulated in liposomes. Liposomes containing the molecule of interest are prepared by methods known in the art, such as described in Epstein et al., Proc. Natl. Acad. Sci. USA 82:3688 (1985); Hwang et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556.

Particularly useful immunoliposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of an antibody can be conjugated to the liposomes as described in Martin et al., J. Biol. Chem. 257:286-288 (1982) via a disulfide interchange reaction to target the liposome. A chemotherapeutic agent (such as Doxorubicin) is optionally contained within the liposome. See Gabizon et al., J. National Cancer Inst. 81(19):1484 (1989).

The formulation herein may also contain more than one active compound, as necessary for the particular indication being treated, preferably those with complementary activities that do not adversely affect each other. Such molecules are suitably present in combination in amounts that are effective for the purpose intended. For example, a p75NTR-Ephrin A agonist may be combined with a chemotherapeutic agent.

The agonists are useful in the treatment of various neoplastic and non-neoplastic diseases and disorders. Cancers and related conditions that are amenable to treatment include breast carcinomas, lung carcinomas, gastric carcinomas, esophageal carcinomas, colorectal carcinomas, liver carcinomas, ovarian carcinomas, thecomas, arrhenoblastomas, cervical carcinomas, endometrial carcinoma, endometrial hyperplasia, endometriosis, fibrosarcomas, choriocarcinoma, head and neck cancer, nasopharyngeal carcinoma, laryngeal carcinomas, hepatoblastoma, Kaposi's sarcoma, melanoma, skin carcinomas, hemangioma, cavernous hemangioma, hemangioblastoma, pancreas carcinomas, retinoblastoma, astrocytoma, glioblastoma, Schwannoma, oligodendroglioma, medulloblastoma, neuroblastomas, rhabdomyosarcoma, osteogenic sarcoma, leiomyosarcomas, urinary tract carcinomas, thyroid carcinomas, Wilm's tumor, renal cell carcinoma, prostate carcinoma, abnormal vascular proliferation associated with phakomatoses, edema (such as that associated with brain tumors), and Meigs' syndrome.

Non-neoplastic conditions that are amenable to treatment include rheumatoid arthritis, psoriasis, atherosclerosis, diabetic and other proliferative retinopathies including retinopathy of prematurity, retrolental fibroplasia, neovascular glaucoma, age-related macular degeneration, thyroid hyperplasias (including Grave's disease), corneal and other tissue transplantation, chronic inflammation, lung inflammation, nephrotic syndrome, preeclampsia, ascites, pericardial effusion (such as that associated with pericarditis), and pleural effusion.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

The formulations to be used for in vivo administration must be sterile. This is readily accomplished by filtration through sterile filtration membranes.

Sustained-release preparations may be prepared. Suitable examples of sustained-release preparations include semipermeable matrices of solid hydrophobic polymers containing the antagonist, which matrices are in the form of shaped articles, e.g., films, or microcapsule. Examples of sustained-release matrices include polyesters, hydrogels [for example, poly(2-hydroxyethyl-methacrylate), or poly(vinylalcohol)], polylactides (U.S. Pat. No. 3,773,919), copolymers of L-glutamic acid and ethyl-L-glutamate, non-degradable ethylene-vinyl acetate, degradable lactic acid-glycolic acid copolymers such as the Lupron Depot. (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated antibodies remain in the body for a long time, they may denature or aggregate as a result of exposure to moisture at 37 C, resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

For therapeutic applications, the antagonists of the disclosure are administered to a mammal, preferably a human, in a pharmaceutically acceptable dosage form such as those discussed above, including those that may be administered to a human intravenously as a bolus or by continuous infusion over a period of time, by intramuscular, intraperitoneal, intracerebrospinal, subcutaneous, intraarticular, intrasynovial, intrathecal, oral, topical, or inhalation routes. The antagonists also are suitably administered by intratumoral, peritumoral, intralesional, or perilesional routes, to exert local as well as systemic therapeutic effects. The intraperitoneal route is expected to be particularly useful, for example, in the treatment of neurological disease or disorder or injury (e.g., mono- and poly-neuropathy) caused by physical, chemical or metabolic injury (e.g., diabetic neuropathy).

For the prevention or treatment of disease, the appropriate dosage of antagonist will depend on the type of disease to be treated, as defined above, the severity and course of the disease, whether the antagonist is administered for preventive or therapeutic purposes, previous therapy, the patient's clinical history and response to the antagonist, and the discretion of the attending physician. The antagonist is suitably administered to the patient at one time or over a series of treatments.

The disclosure also provides methods of screening an agent for p75NTR-ephrin A agonist and antagonist activity. In one embodiment the agent is contacted with a cell capable of developing a p75NTR-ephrin A complex and measuring Fyn phosphorylation.

Based upon the foregoing and the following specific examples, the disclosure demonstrates that inhibition of ephrinA-p75NTR complex of the activation of p75NTR can modulate the reverse signaling associate with complex formation and p75NTR activation and phosphorylation of Fyn. Inhibiting the reverse signaling promote axonal development and provides methods for treating neuronal damage requiring axonal development. Furthermore, it will be recognized based on the present disclosure that stimulating reverse signaling can inhibit cytoskeletal development thereby treating the cytoskeletal development associated with various metastatic diseases and disorders. Thus promoting or stimulating p75NTR activity or the formation of the p75NTR-ephrin A complex provides a method of treating metastasis by promoting reverse signaling in such cells.

As demonstrated further below, reverse signaling through ephrin-As on RGC axons is implicated in the development of the retinocollicular map (Rashid et al., 2005) and in several other axonal projections (Knoll et al., 2001; Cutforth et al., 2003; Marquardt et al., 2005). However, because ephrin-As are GPI-linked proteins and lack an intracellular domain, they require a transmembrane signaling partner to initiate the intracellular pathways that carry out their functions. The disclosure shows that p75NTR is a signaling partner for ephrin-As and activates an intracellular cascade that mediates the repellent effects of ephrin-A reverse signaling on RGC axons required for their proper guidance and mapping.

The experiments presented herein demonstrate p75 is expressed in RGCs and that p75 protein is present in their axons in vivo at the appropriate developmental stages to mediate guidance and mapping. In addition, p75 co-localizes with ephrin-As along retinal axons and complexes with ephrin-As in caveolae. Further, this association of p75 and ephrin-A results in a functional signaling complex that when activated by EphA binding to ephrin-As leads to increased levels within caveolae of phosphorylated Fyn. The demonstration that EphA binds ephrin-A but not p75 indicates that EphAs are not ligands per se for p75, but through its association with ephrin-As, p75 acts as co-receptor, or signaling partner, for them and is required to activate their reverse signaling pathway. Phosphorylation was also increased and recruitment of Fyn to caveolae is dependent upon p75, which itself is recruited to caveolae upon EphA binding ephrin-A.

A p75NTR polypeptide can comprise a sequence having 70, 80, 90%, or more identity to the following sequence:

| | |
|---|---|
| 1 | mgagatgram dgprllllll lgvslggake acptglyths gecckacnlg egvaqpcgan |
| 61 | qtvcepclds vtfsdvvsat epckpctecv glqsmsapcv eaddavcrca ygyyqdettg |
| 121 | rceacrvcea gsglvfscqd kqntvceecp dgtysdeanh vdpclpctvc edterqlrec |
| 181 | trwadaecee ipgrwitrst ppegsdstap stqepeappe qdliastvag vvttvmgssq |
| 241 | pvvtrgttdn lipvycsila avvvglvayi afkrwnsckq nkqgansrpv nqtpppegek |
| 301 | lhsdsgisvd sqslhdqqph tqtasgqalk gdgglysslp pakreevekl lngsagdtwr |
| 361 | hlagelgyqp ehidsfthea cpvrallasw atqdsatlda llaalrriqr adlveslcse |
| 421 | statspv |

see also accession no: NP_{—}002498 incorporated herein by reference. Using the foregoing information one of skill in the art can identify the cytoplasmic domain, coding sequence and develop siRNA inhibitors.

Accordingly, soluble domains of p75 can be used as antagonists as described above with respect to Ephrin A. For example, a soluble domain of P75NTR can be operably linked to an Fc, leucine zipper or linker to form oligomers. The oligomers can compete with the natural ligand for p75NTR thereby preventing the activity of the ligand in vivo.

A large number of Ephrin A polypeptide sequences are known in the art and are incorporated herein by reference. One of skill in the art can readily identify sequences by search the National Center for Biotechnology Information Entrez Database.

The following examples are intended to illustrate but not limit the disclosure. While they are typical of those that might be used, other procedures known to those skilled in the art may alternatively be used.

### EXAMPLES

Immunohistochemistry. Anesthetized mice were perfused with 4% paraformaldehyde (PF), dissected, and cryoprotected in 30% sucrose. Cryostat sections (20 μm) were processed with antibodies and receptor affinity probes from R&D Systems (anti-ephrin-A2, AF603; anti-ephrin-A5, AF3743; ephrin-A5-Fc, 374-EA) and Santa Cruz (anti-p75, sc-6188; Brn3.2, sc-6026). Some retinal sections were labeled with anti-GFP antibodies (Molecular Probes, A11122). Retinal axons and 293 cells grown in vitro were fixed with 4% PF in PBS for 10-15 minutes, washed, and processed with the reagents above as well anti-Fc antibody (Jackson Immuno, 309-166-008) and EphA7-Fc (R&D Systems, 608-A7).

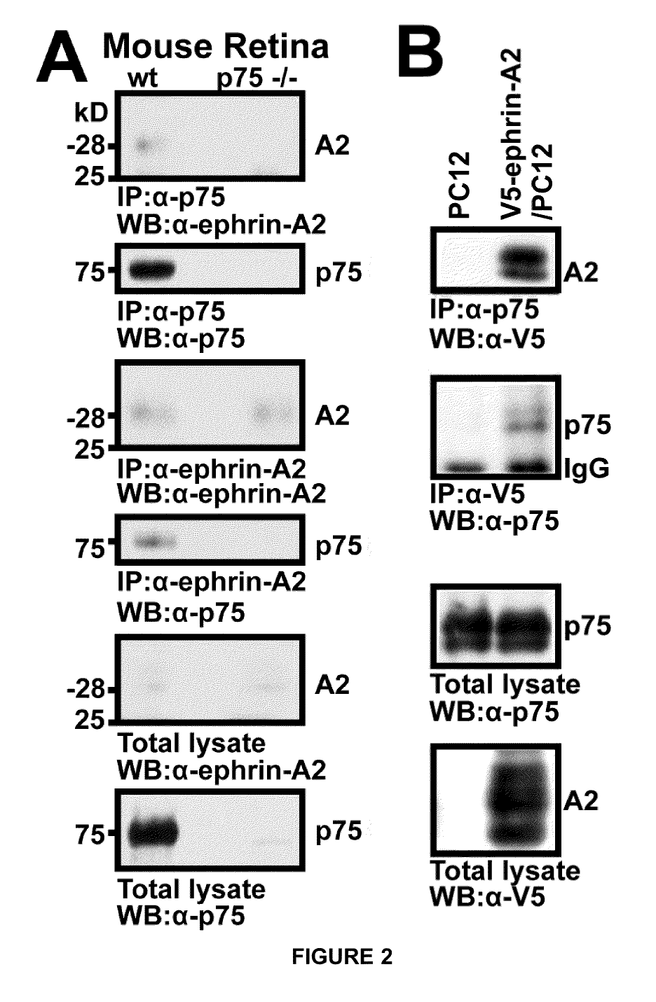

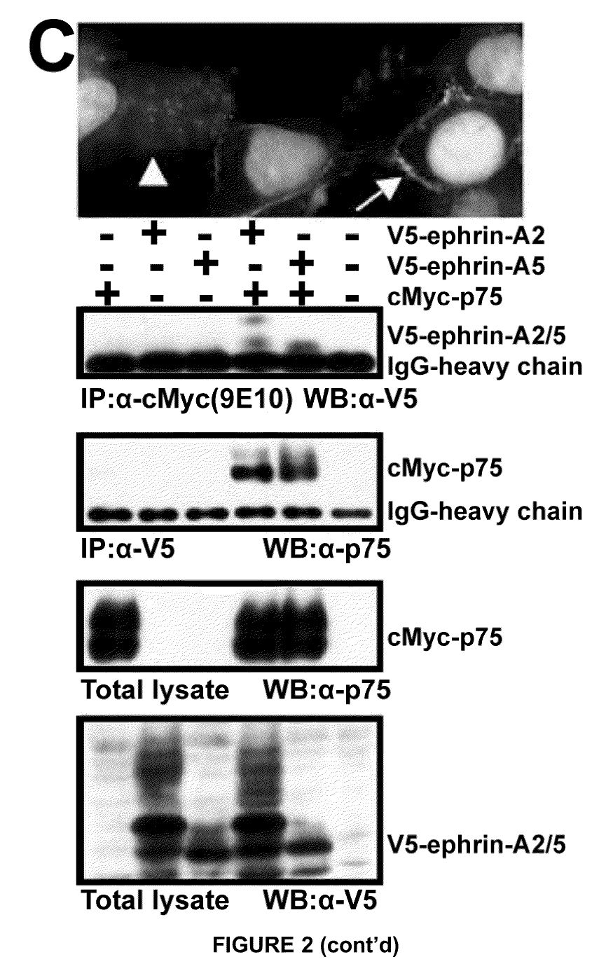

Immunoprecipitation. For FIG. 2A, mouse retinas were lysed in RIPA buffer (150 mM NaCl, 1% NP-40, 0.5% DOC, 0.1% SDS, 50 mM Tris pH 8.0). Lysates were immunoprecipitated with anti-p75 intracellular domain antibody (Buster, a gift from Philip A. Barker, McGill University) or anti-ephrin-A2 antibody (R&D Systems, AF603). Immunoprecipitations were performed using ExactaCruz™ F and C kits (Santa Cruz, sc-45043 and sc-45040) to decrease IgG bands. Samples were analyzed using SDS-PAGE and Western blots. For detection of p75 and ephrin-A2, anti-p75 antibody (Buster) and anti-ephrin-A2 antibody (Santa Cruz, L-20, sc-912) were used, respectively. For FIG. 2B, PC12 cells were transfected with linearized V5-tagged ephrin-A2 construct by TransFectin™ lipid reagent (Biorad, 170-3352) and selected by puromycin (2□g/ml) for at least 14 days. Colonies were picked and characterized by immunocytochemistry and Western blots. PC12 and stably transfected V5-ephrin-A2/PC12 cells were lysed in RIPA buffer and immunoprecipitated with either anti-p75 (Buster) or anti-V5 antibody (Invitrogen, R960-25). For detection of p75 and V5-ephrin-A2, anti-p75 antibody (Buster) and anti-V5 antibody (Invitrogen) were used. For FIG. 2C, 293T cells were transiently transfected with cMyc-tagged p75 and/or V5-tagged ephrin-A5 or ephrin-A2 constructs by TransFectin™. Cells were lysed in RIPA buffer, and immunoprecipitated with either anti-cMyc antibody (Santa Cruz, 9E10, sc-40) or anti-V5 antibody (Invitrogen). For detection of p75 and V5-ephrin-A2/5, anti-p75 antibody (Buster) and anti-V5 antibody (Invitrogen) were used.

Isolation of caveolae. Stably transfected 293 cell lines (ephrin-A2, p75 and ephrin-A2/p75) were made by transfecting with linearized V5 tagged ephrin-A2 and/or cMyc tagged p75 constructs using TransFectin™ and selected by puromycin (2 μg/ml) and/or G418 (400□g/ml) for at least 14 days. Colonies were picked and characterized by immunocytochemistry and Western blots. Detergent-resistant membrane fractions containing caveolae were prepared by modifying a procedure originally described by Higuchi et al. (2003). Cells were grown to 90% confluence and serum-starved for 18 hours before treatment. Cells were treated with either human-Fc (R&D systems, 110-HG, 2 μg/ml) or EphA7-Fc (R&D Systems, 608-A7, 2 μg/ml) for 10 minutes at 37° C., then lysed on ice with 1 ml of 0.5% Brij-58 (Sigma) in buffer containing 10 mM Tris pH 7.5, 1 mM EDTA, 150 mM NaCl, 10% glycerol, phosphatase inhibitor cocktail I and II, and protease inhibitor cocktail (Sigma). Lysed cells were scraped from plates into individual tubes and kept on ice for 30 minutes. Lysates were adjusted to 40% sucrose, 2 ml of the mixture was placed in the bottom of an SW41Ti ultracentrifuge tube (Beckman), and overlaid with 8 ml of 30% sucrose and 2 ml of distilled water. All steps from lysis to centrifugation were performed at 4° C. After centrifugation (16 h, 35,000 rpm, 4° C.), 1 ml fractions were collected from the top to the bottom (numbered from 1 to 12). The proteins in each fraction were precipitated for concentration and sucrose removal. Briefly, 600□l methanol was added to 150 μl of each fraction. After thorough mixing, 150 μl of chloroform was added. After vortexing, 450 μl of water was added, vortexed again, and centrifuged for 5 minutes at full speed in a microcentrifuge. The upper aqueous layer was discarded, 650□l of methanol added, and each tube was inverted 3 times. After 5 minutes at full speed in a microcentrifuge, all liquid was removed and the pellets were air-dried. Equal volume of Laemmli's sample buffer (30 μl) was added, samples were heated at 100° C. for 5 minutes, and prepared for SDS-PAGE and Western blot analyses. For detection of tyrosine phosphorylation, Fyn, p75, V5-tagged ephrin-A2, flotillin-1 and GM1, phosphotyrosine-specific antibody 4G10 (a gift from Tony Hunter, Salk Institute), anti-Fyn antibody (Santa Cruz, sc-16), anti-p75 intracellular domain antibody (Buster), anti-V5 antibody (Invitrogen, R960-25), anti-flotillin-1 antibody (BD Transduction Laboratories, 610820) and CTX-HRP (Invitrogen, C34780) were used.

Mice. p75 null mutants were described previously (Lee et al. 1992). To generate p75 conditional mutants, two LoxP sites were introduced into the p75 locus to flank exon 3 through homologous recombination in embryonic stem cells (Z. Chen, T-C. Sung, N. Harada, W. Lin and K-F. Lee, in preparation). p75 conditional mutants were crossed with α-cre transgenic mice (Marquardt et al., 2001). In some cases the ROSA-GAP43-eGFP allele was also present.

Stripe assays. Retinas from P0-P2 mice were dissected, flattened onto a nitrocellulose filter, and cut into strips 150-400 μm wide. Strips were plated, RGC side down, onto glass coverslips or plastic dishes coated with alternating stripes of human-Fc or EphA7-Fc and human-Fc or ephrin-A5-Fc and human-Fc (R&D Systems 110-HG; 15-30 μg/ml) and laminin. Stripes were made essentially as described (Hornberger et al., 1999; Rashid et al., 2005). After 2-4 days, explants were stained with carboxyfluorescein diacetate, succinimidyl ester (fluorescent vital dye; Molecular Probes), examined, photographed, and scored independently by two investigators blind to experimental condition, lane content, and genotype of the explant source. A score of zero indicates no discernible choice; one indicates any detectable bias; two indicates a clear bias; three indicates a strong and significant choice for a significant majority of axons; four indicates an essentially complete choice for one lane. Pixel values were determined by thresholding each grayscale photo using Adobe Photoshop until pixels representing axons were white and background was black. Pixels clearly representing debris were converted to background. Pixels were counted in each lane and normalized for lane width. The modified Sholl intersection analysis was performed by delineating the edge of each explant and points 100 μm, 300 μm, 600 μm, 900 μm away. Blind to genotype, lane condition and lane position, all intersections between axons and the transposed explant outlines were digitally marked. With all intersections marked, lane boundaries were overlaid and the position of each intersection point was assigned to a lane and normalized for lane width. The coefficient of choice is defined as the total pixels representing axons or intersections on control lanes minus that on the second human-Fc lane, or the EphA7-Fc or ephrin-A5-Fc lanes, divided by total pixels or intersections. A coefficient of one indicates an absolute choice for the control lane, a coefficient of zero indicates no choice, and a negative coefficient of choice indicates a choice for the EphA7-Fc or ephrin-A5-Fc lane.

Axon tracing and analysis. Focal injections of the lipophilic, fluorescent axon tracer DiI (Molecular Probes) were made via pressure injection through a glass micropipette tip into the retina and allowed to transport for 16-24 hours. Mice were perfused, dissected and axon labeling photographed. Some midbrains were sectioned on a vibratome at 100-200 μm. The focal nature and fidelity of all injections was determined by retinal flat-mounts examined under fluorescence. All labeled axons originated from a single focal location in every case reported.

Analyses of TZ position, DiI injection location, and eGFP domains in the SC were performed on digital images in Adobe Photoshop or NIH ImageJ software and analyzed with Excel or KaleidaGraph software. The center of the DiI injection and TZ were used for the analyses and determined to be the center of a circumscribed circle. For the analysis of eGFP-labeled projection domains, the SC was divided into 10 equal segments along the LM axis. The anterior and posterior borders of the central domain were determined in each segment by thresholding at three times the average pixel value of an arbitrarily selected area within the central domain of that segment. Segments were combined and pixel values counted in the three defined domains. Values for domain sizes are normalized. Total SC area is not statistically different between genotypes.

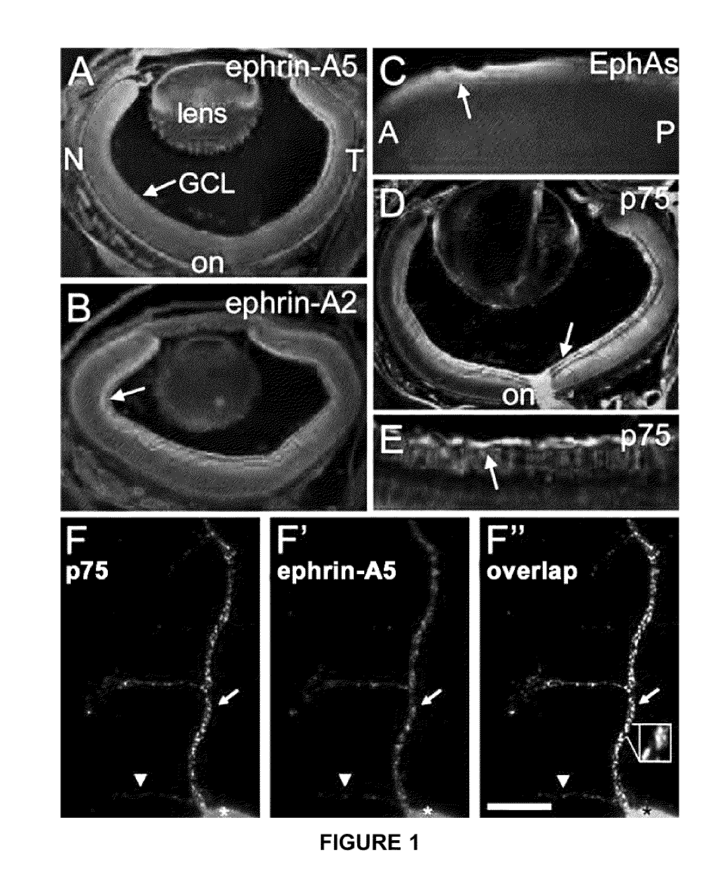

Distributions of EphAs, ephrin-As, and p75 in developing retinocollicular projection. Previous reports have demonstrated the expression of ephrin-As in gradients in the embryonic and postnatal retina (McLaughlin and O'Leary, 2005). In addition, multiple EphAs are expressed in gradients in the SC (Rashid et al., 2005). For the purpose of these experiments, protein distribution of ephrin-As in the retina and EphAs in the SC at P2 were analyzed, the midpoint in development of retinotopic map in the SC. Immunostaining for ephrin-A5 and ephrin-A2 reveals that each is expressed in a high-to-low NT gradient in the retina, including by RGCs (FIGS. 1A and 1B). The gradient of ephrin-A5 is steep and restricted primarily to nasal retina, whereas the ephrin-A2 gradient is shallow but extends across most of the NT retinal axis. In addition, both ephrin-A5 and ephrin-A2 are present along RGC axons as they exit the retina and form the optic nerve (FIG. 1A).

To determine the distribution of EphA protein in the SC at P2, an ephrin-A5-Fc affinity probe was used that binds to all EphAs and detected by Fc-specific antibodies (FIG. 1C). EphAs are shown to b distributed in an overall high-to-low AP gradient in superficial layers of the SC where RGC axons navigate across the AP axis of the SC as well as arborize.

p75 is expressed by RGCs throughout development of the retinocollicular projection (Harada et al., 2006). By specific immunostaining, at P2 p75 protein is distributed across the retina with no obvious gradient, and is present in RGCs and along RGC axons (FIGS. 1D and 1E). Similar distributions of p75 protein at E16 were found, when the first RGC axons reach the SC, through at least P8, when the retinocollicular map resembles its mature form. Thus, p75 is distributed along RGC axons at the appropriate time to mediate ephrin-A reverse signaling during their guidance and mapping.

To investigate co-localization of p75 and ephrin-A proteins along RGC axons, immunohistochemistry was performed on primary cultures of dissociated mouse retina. A punctate distribution of both p75 and ephrin-A5 in microdomains that resemble caveolae is found along the primary axon shaft, branches, and growth cones of RGCs (FIGS. 1F and 1F′). Domains of p75 often co-localize with domains of ephrin-A5 and ephrin-A2, though non-overlapping domains are also evident (FIG. 1F″). This co-localization of p75 and ephrin-As in caveolae-like domains along RGC axons is consistent with the distribution required for their biochemical association.

p75 associates with ephrin-As and is required for their reverse signaling. To establish whether p75 and ephrin-A's associate in protein complexes, a series of immunoprecipitation assays were carried out. Incubating tissue prepared from mouse retina with antibodies against either p75 or ephrin-A2 results in the co-immunoprecipitation of ephrin-A2 and p75, respectively (FIG. 2A). PC12 cells were also analyzed, which endogenously express p75, stably transfected with ephrin-A2 linked to the V5 epitope (V5-ephrin-A2). Immunoprecipitation using an antibody specific for p75 (the “Buster” intracellular domain antibody) pulls down V5-ephrin-A2, and a V5 antibody for the tagged ephrin-A2 pulls down p75 (FIG. 2B). The distributions of ephrin-As and p75 were also analyzed on transfected cells in vitro. In 293 cells transfected with cMyc-tagged p75 or V5-ephrin-A5, EphA7-Fc binds only those cells expressing ephrin-A5, indicating that EphA7 itself does not bind p75 extracellularly (FIG. 2C). In addition, ephrin-A5 and p75 are found in discrete caveolae-like puncta on the cell membrane, similar to their distributions along RGC axons. Immunoprecipitations and western blots performed on 293T cells transiently co-transfected with cMyc-p75 and either V5-ephrin-A5 or V5-ephrin-A2 using antibodies directed against either the cMyc or V5 epitopes co-immunoprecipitate p75 with either ephrin-A2 or ephrin-A5 (FIG. 2C). Thus, these findings using cell lines corroborate those obtained with mouse retina, and together suggest that ephrin-As and p75 are present as a protein complex in the membrane. This association between p75 and ephrin-As, together with their co-localization in discrete domains along RGC axons, suggest functional implications for p75-ephrin-A complexes in activating intracellular signaling in response to EphAs that controls RGC axon guidance and mapping.

Fyn has been implicated in ephrin-A reverse signaling (Davy et al., 1999). Therefore, experiments were performed to determine whether p75 is involved in the Fyn signaling pathway associated with ephrin-A reverse signaling. EphA7-Fc binds ephrin-As but does not bind p75 on the surface of transfected 293 cells (FIG. 2C), indicating that EphA7 is not a ligand for p75 but is a ligand for ephrin-A. Although p75 does not bind EphAs, the data herein show that the association of p75 with ephrin-A is required for ephrin-A reverse signaling,

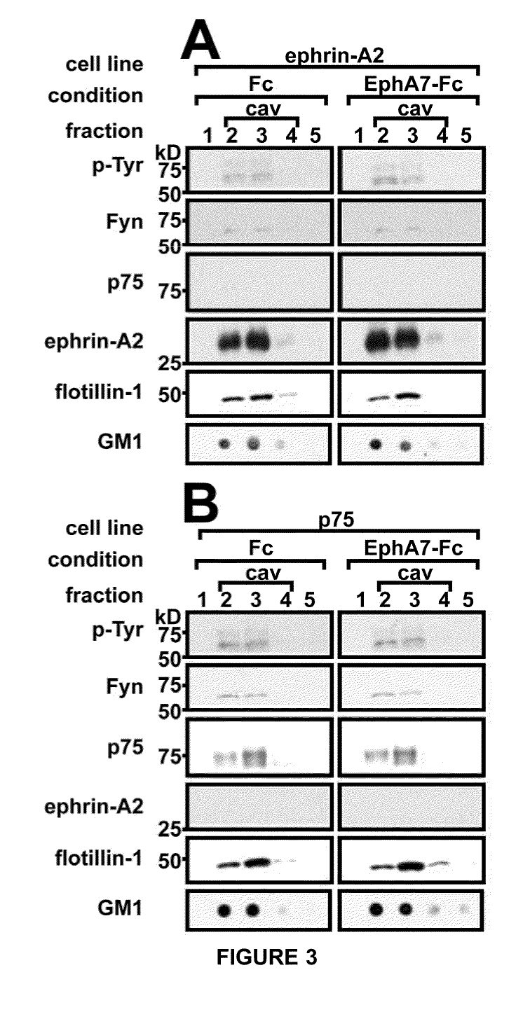

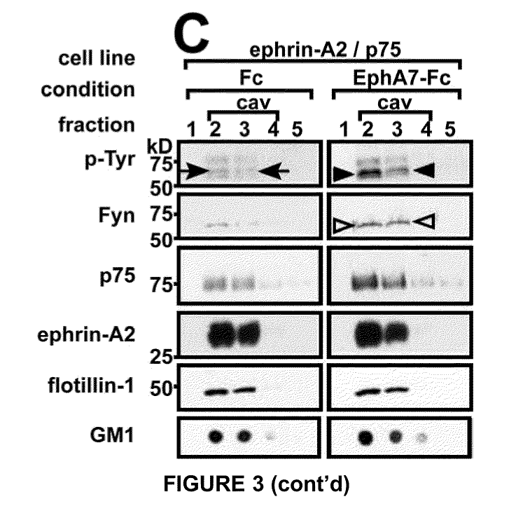

The phosphorylation of Fyn in stably transfected 293 cells was then examined to determine if Fyn phosphorylation associated with ephrin-A reverse signaling is dependent upon p75. Previous studies of ephrin-A reverse signaling in transfected cell lines have shown that the enhanced phosphorylation of Fyn occurs predominantly in the caveolae fraction with a very minor increase in the soluble fraction (Davy et al., 1999). These findings are consistent with the preferential localization of ephrin-As and p75 to caveolae (Davy et al., 1999; Higuchi et al., 2003; present study). Therefore, caveolae containing fractions isolated from stably transfected 293 cells and identified with antibodies against the caveolae-specific protein, flotillin-1 (Higuchi et al., 2003; Slaughter et al., 2003) were examined and the presence of the caveolae-specific lipid, GM1 (Parton, 1994; FIG. 3). EphA7-Fc was used to stimulate ephrin-A reverse signaling by its binding of ephrin-A, and as a control for this stimulation, Fc alone was used. These are the same proteins used in the protein stripe assay, described herein, to show that the repellent effect of ephrin-A reverse signaling on retinal axons is dependent upon p75.

The data demonstrate that cells stably transfected with either ephrin-A2 or p75, the level of phosphorylated Fyn or even overall phosphotyrosine levels do not change after treatment with EphA7-Fc compared to Fc treatment (FIGS. 3A and 3B). However, when both ephrin-A2 and p75 are present, a significant increase in the overall level of both phosphotyrosine and Fyn after treatment with EphA7-Fc was found, compared to treatment with Fc, in the caveolae fractions (n=2; FIG. 3C). These data indicate that p75 complexes with ephrin-As and is required for activation of ephrin-A reverse signaling through an intracellular pathway involving Fyn. Interestingly, the level of p75 is increased in the caveolae fractions following EphA7-Fc treatment, compared to Fc treatment, in cells in which ephrin-A2 is also present. In addition, to the substantial increase in phosphotyrosine level induced by EphA, the findings that the recruitment of p75 and Fyn to caveolae is also dependent on EphA binding ephrin-A, supports their involvement in ephrin-A reverse signaling.

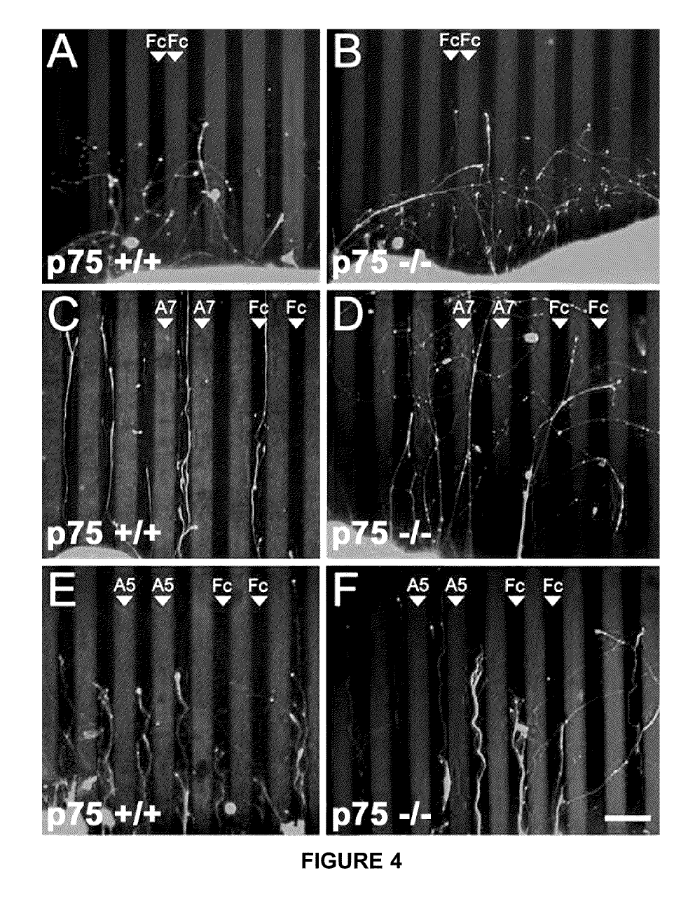

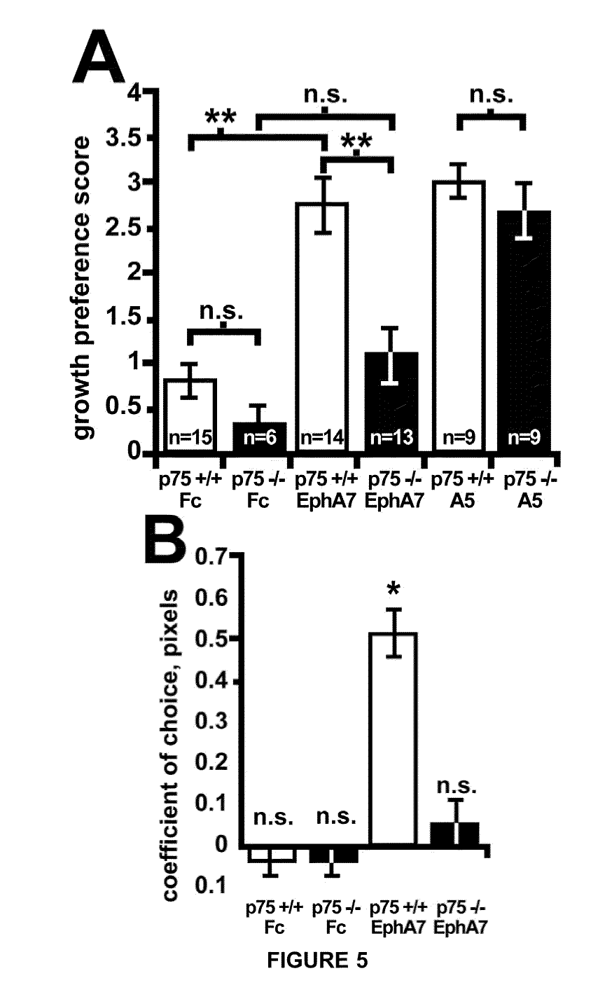

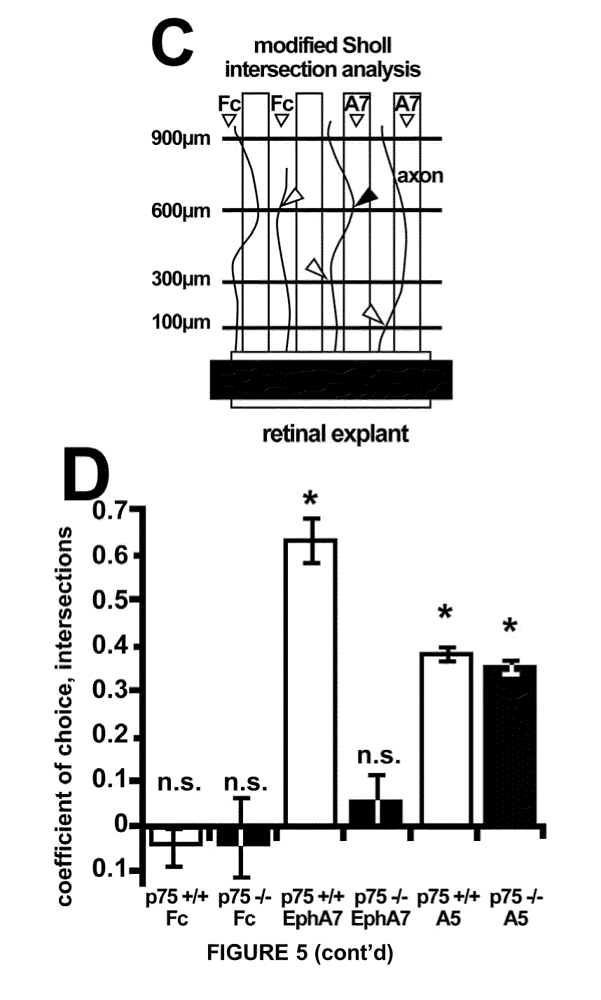

Repellent effect of EphA7 on retinal axons requires p75. The protein stripe assay was used to assess whether the repellent activity of ephrin-A reverse signaling for retinal axons requires their expression of p75. Axons extending from retinal explants from P0 wild type (p75^{+/+}) and p75 knockout mice (p75^{-/-}; Lee et al., 1992) were given a choice to grow on alternating stripes of EphA7-Fc and Fc, or in control experiments, alternating stripes that each contain Fc (FIG. 4). EphA7 was chosen because it is expressed in a high to low AP gradient in the SC and repels wild type retinal axons in the protein stripe assay (Rashid et al., 2005). In control experiments, neither p75^{+/+} nor p75^{-/-} retinal axons exhibit a growth preference for either set of Fc stripes (FIGS. 4A and 4B). However, when given a choice between alternating stripes of EphA7-Fc and Fc, p75^{+/+} retinal axons demonstrate a strong preference for stripes containing Fc and a strong avoidance of stripes containing EphA7-Fc (FIG. 4C). In contrast, p75^{-/-} retinal axons do not exhibit a significant preference for either the EphA7-Fc or Fc set of stripes and instead have similar outgrowth on each set (FIG. 4D). These qualitative impressions of the growth preferences are supported by three distinct quantitative methods, all performed blind to genotype and stripe content, that include the classic method of scoring growth preference on a scale from 0-4 (FIG. 5A; Walter et al., 1987), quantification of pixels representative of stained axons on each set of stripes (FIG. 5B), and a modified Sholl intersection analysis (Sholl, 1953; FIGS. 5C and 5D).

Additional stripe assay experiments were performed to assess potential effects of p75 deficiency on the repellent effect of EphA forward signaling in response to ephrin-As exhibited by retinal axons. These experiments were carried out as described above except EphA7-Fc was replaced with ephrin-A5-Fc. p75^{+/+} retinal axons preferentially avoid ephrin-A5 containing stripes (FIG. 4E), consistent with previous reports (e.g. Feldheim et al., 1998) and that p75^{-/-} retinal axons also show a strong avoidance of ephrin-A5 stripes (FIG. 4F) indistinguishable from wild type (FIGS. 5A and 5D). Therefore, p75^{+/+} and p75^{-/-} retinal axons exhibit a similar repellent response to ephrin-A5 mediated by EphA forward signaling.

In additional experiments, the concentration of ephrin-A5-Fc was titrated in the stripes to the level at which p75^{+/+} retinal axons exhibit a small but significant preference for the Fc set of stripes (coefficient of choice=0.22; p<0.05; n=9). Matched sets of retinal explants from p75^{-/-} mice grown on the same substrates in the same dish as the retinas from p75^{+/+} littermates exhibit a similar degree of preference for the Fc stripes (coefficient of choice=0.20; p<0.05; n=9; coefficients of choice are not significantly different from each other). Thus, p75^{+/+} and p75^{-/-} retinal axons exhibit the same degree of repulsion to ephrin-A5 at both high and low concentrations of the repellent activity. These data indicate that p75^{-/-} retinal axons are not only repelled by ephrin-A5 to a similar degree as p75^{+/+} retinal axons, but that both exhibit the same sensitivity to ephrin-A5. Thus, p75 deficiency does not significantly influence EphA forward signaling in retinal axons or mechanisms required for them to exhibit a repellent response.

p75^{+/+} and p75^{-/-} retinal axons do not exhibit significant differences in general outgrowth. For example, over all of the stripe experiments described, p75^{+/+} retinal explants extend on average 26 axons, similar to p75^{-/-} retinal explants that extend 25 axons (n=47 for p75^{+/+}; n=37 for p75^{-/-}; n.s.). Similarly, the extent of axon growth is similar between genotypes. For example, approximately 27% of all axons that extend at least 100 μm from a retinal explant also extend 900 μm for both p75^{+/+} and p75^{-/-} retinas (n=1237 axons for p75^{+/+}; n=933 axons for p75^{-/-}; n.S).

Thus, the avoidance of EphA7-Fc by p75^{-/-} retinal axons is not due to a general inability to extend or respond to guidance cues, but rather is due to a specific defect in ephrin-A reverse signaling. Taken together, these data show that p75 mediates the repellent effect of EphAs on retinal axons, and together with our biochemical and co-localization data, strongly suggest that p75 is a signaling partner for ephrin-A reverse signaling.

Use of p75 mutant mice to study requirement for p75 in retinotopic mapping. The findings that p75 is required in vitro for the repulsion of retinal axons by ephrin-A reverse signaling shows that p75 is required for the proper development of the retinotopic map in the SC. Thus, repulsion of RGC axons mediated by ephrin-A reverse signaling will be diminished, resulting in an anterior shift of their projections. To confirm this, complementary axon tracing methods were used to analyze the topographic organization of the retinocollicular projection in constitutive p75 knockout mice (Lee et al., 1992) and in conditional p75 knockout mice in which floxed (fl) alleles of p75 were selectively deleted from RGCs localized to specific retinal domains. One labeling method is anterograde labeling of a small number of RGC axons by a small focal injection of the lipophilic axon tracer DiI in the retina. The other method labels, in a reproducible manner, large domains of RGCs and their axonal terminations using a conditional eGFP marker and the α-cre recombinase allele that is expressed in nasal and temporal retina, but not in central retina or along the visual pathway (Marquardt et al., 2001; Baumer et al., 2002).

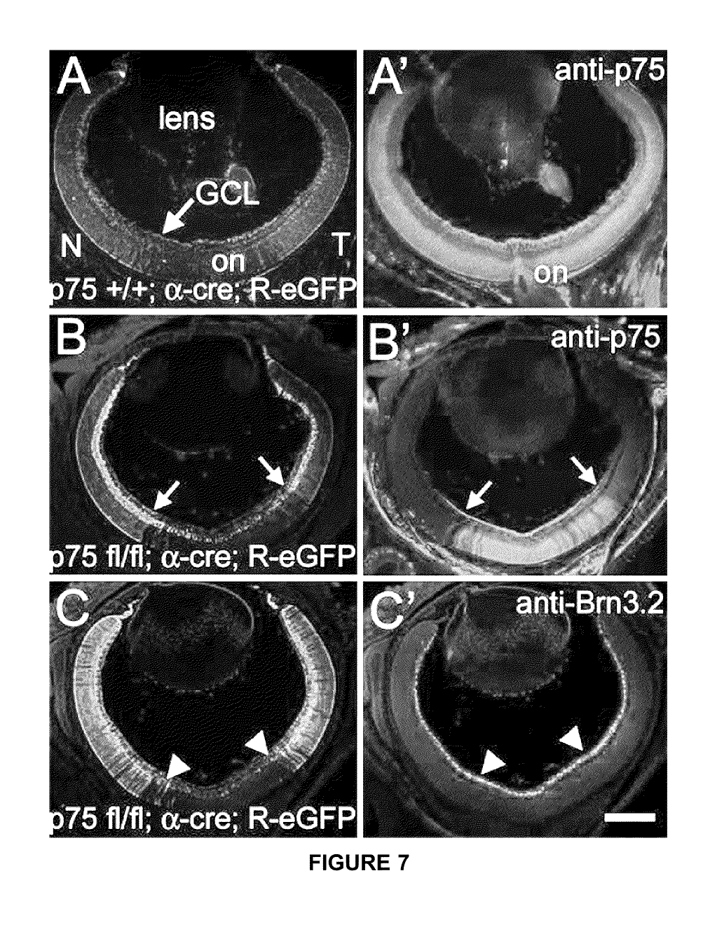

The development, size and patterning of the retina are normal in the constitutive and conditional p75 mutants. Markers specific for RGCs (e.g. Brn3.2; FIGS. 7C and C′; Xiang et al., 1993), as well as general cell stains, reveal that at late embryonic and postnatal ages the size and laminar patterning of the retina, and density of RGCs, is indistinguishable between p75^{-/-} mice and their p75^{+/+} littermates (Harada et al., 2006). Further, these genotypes have no difference in the graded expression of ephrin-A2 and ephrin-A5, indicating that expression of axon guidance molecules and axial patterning of the retina is normal in the absence of p75.

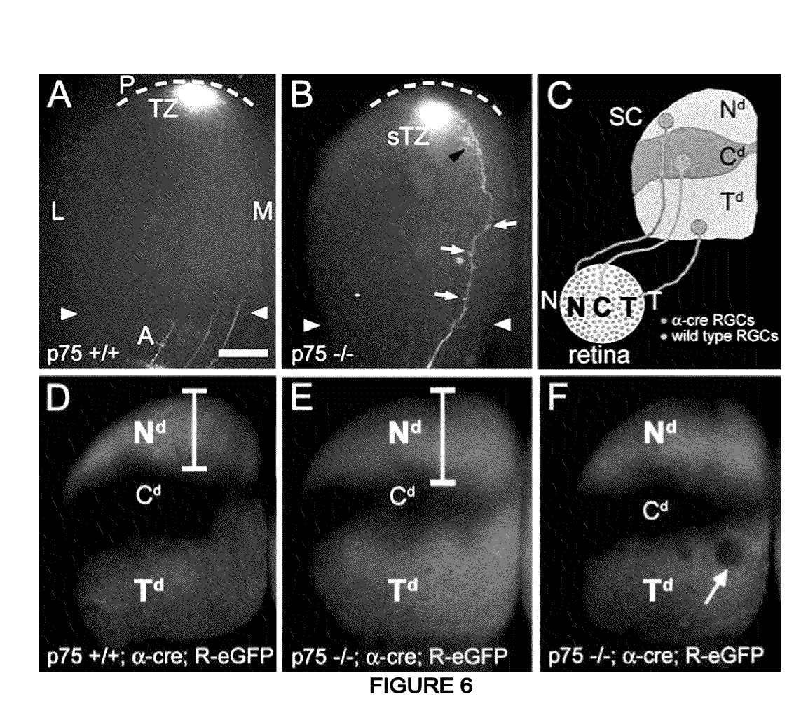

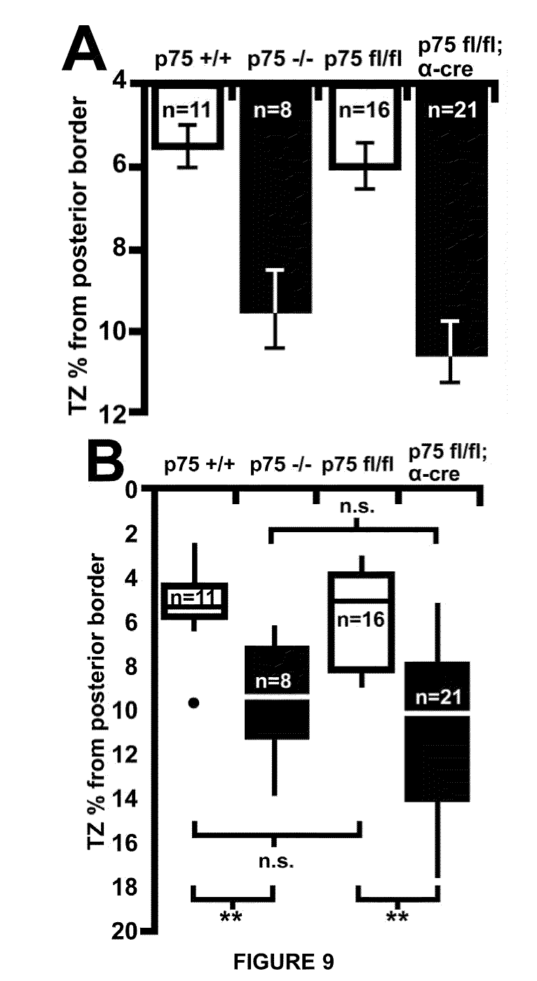

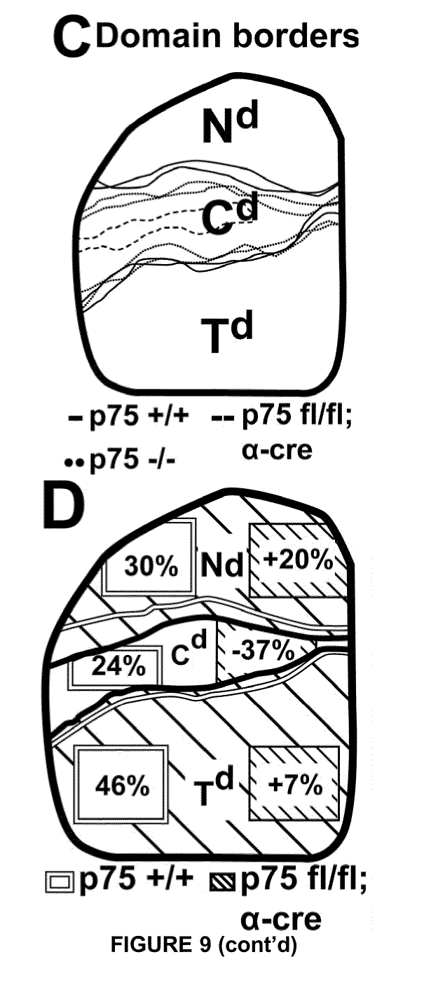

Analyses of constitutive null p75 mice. The topographic organization of the retinocollicular projection were analyzed in constitutive null p75 mice and their p75^{+/+} littermates by making a small focal injection of DiI into peripheral nasal retina. At neonatal stages, prior to map refinement, the projections labeled in p75^{+/+} and p75^{-/-} mice are indistinguishable, including the degree of axon overshoot. By P8, when the map is normally properly ordered (Frisen et al., 1998), a nasal DiI injection in p75^{+/+} mice labels a dense, focal TZ in the topographically appropriate position in posterior SC (n=11; FIG. 6A). A similar nasal injection of DiI in p75^{-/-} mice results in a dense, focal TZ, but in every case the TZ is shifted anteriorly compared to its position in p75^{+/+} littermates (n=8; FIG. 6B). Quantification shows that the positions of the DiI injection sites on the TN retinal axis are not statistically different between genotypes, but in contrast the anterior shift of the TZ formed by nasal RGC axons on the AP SC axis in p75^{-/-} mice compared to p75^{+/+} littermates is statistically significant (FIG. 9A). Additionally, the AP position of the TZ in each p75^{-/-} case is positioned anterior to the mean TZ position for p75^{+/+} cases (FIG. 9B).

In addition to an anterior shift in the TZ in p75^{-/-} mice, in half of the p75^{-/-} cases, ectopic branches and arbors are present along the AP length of nasal axons in the SC at P8 (FIG. 6B), an age when the retinotopic map in p75^{+/+} mice is refined to its mature form and branches are not observed outside the TZ.

To study the organization of the retinocollicular map at a population-level, the α-cre line, which expresses cre recombinase in nasal and temporal retina but not in central retina (Marquardt et al., 2001; Baumer et al., 2002), was crossed to the ROSA-GAP43-eGFP line (R-eGFP) that requires cre-mediated deletion of a floxed-stop cassette to express an eGFP reporter under control of the ROSA promoter (Sapir et al., 2004). This strategy selectively labels nasal and temporal retina, including RGC axons and their terminations within the SC (FIG. 6C). This α-cre; R-eGFP compound line was crossed with p75 mutant mice to study more broadly the effect of p75 deletion on retinotopic mapping.

In p75^{+/+}; α-cre; R-eGFP mice, the projections of RGCs in the nasal and temporal domains of retina are labeled by eGFP, revealing the retinotopic pattern of their terminations, that include a nasal domain (N^{d}) in posterior SC and a temporal domain (T^{d}) anterior SC, respectively, as well as an eGFP negative central domain (C^{d}) formed by the axonal terminations of RGCs in central retina that do not express cre (n=10; FIGS. 6C and 6D). However, in p75^{-/-}; α-cre; R-eGFP mice, the N^{d} of this termination pattern formed by eGFP positive nasal RGC axons shows a significant anterior expansion into the eGFP negative C^{d} formed by eGFP negative central RGC axons (n=7; FIGS. 6E and 6F). Further, in a subset of these p75^{-/-} cases, regions of lower eGFP expression are evident in the T^{d} (FIG. 6F). These regions of diminished eGFP expression are not observed in p75^{+/+} littermates, indicating a level of disorganization in the retinotopic map of p75^{-/-} mice that likely reflects ectopic arborizations formed by eGFP-negative central RGC axons, consistent with DiI labeling in p75^{-/-} mice.

To quantify these mapping changes in p75 mutants, the relative mean areas occupied by each of the three projection domains (N^{d}, C^{d}, and T^{d}) in the SC were measured (FIG. 9). Compared to p75^{+/+} mice, the N^{d} shows a significant increase in area in the p75^{-/-} mice (13% increase, p<0.02), confirming an anterior shift in the TZs of p75 deficient nasal axons. Consistent with this anterior shift, the C^{d} is diminished in size in p75^{-/-} mice compared to p75^{+/+} mice (27% decrease, p<0.02). In summary, in p75^{-/-} mice, the terminations of nasal RGC axons in the SC are shifted anteriorly to those in p75^{+/+} mice. The total area of the SC is not significantly different between genotypes, thus these differences in the sizes of the termination domains are both relative and absolute. This anterior shift of the terminations of RGC axons is consistent with a diminished repellent effect of ephrin-A reverse signaling due to their loss of the ephrin-A signaling partner, p75.

Analyses of mice with retina specific deletion of floxed alleles of p75. To further study the influence of p75 on retinotopic mapping in vivo, mice with a conditional allele (floxed, fl) of p75 that can be removed by cre recombinase were used. For wild type controls mice containing the p75 floxed allele but not the α-cre allele (p75 fl/fl or p75 fl/+ and cre negative) and p75^{+/+}; α-cre were used; R-eGFP mice, none of which affect retinocollicular development or mapping. In p75^{+/+}; α-cre; R-eGFP mice, the nasal and temporal retina are labeled by eGFP without affecting p75 expression (FIGS. 7A and 7A′). In p75 fl/fl; α-cre; R-eGFP mice, eGFP is also expressed in nasal and temporal retina but p75 protein is also selectively eliminated; cells in central retina lack cre recombinase and therefore do not express eGFP and retain wild type levels of p75 protein (FIGS. 7B and 7B′). This altered pattern of p75 protein distribution in p75 fl/fl; α-cre mice is evident prior to E16, when RGC axons first reach the SC, and thereafter.

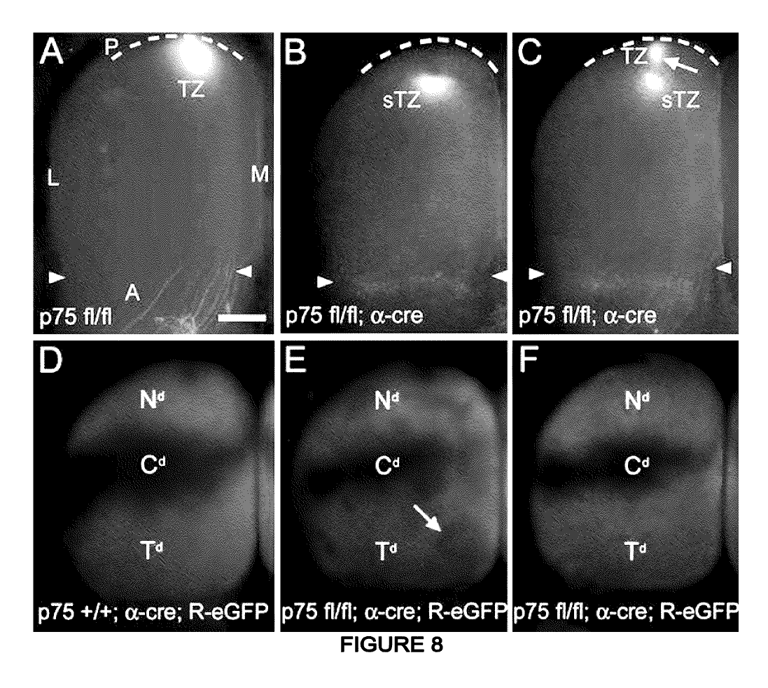

Nasal RGC axons in p75 fl/fl; α-cre mice exhibit similar phenotypes as in p75^{-/-} mice (FIG. 8). Focal injections of DiI in nasal retina of p75 fl/fl; cre-negative mice at P8 (n=16) reveal a projection indistinguishable from wild type mice (FIG. 8A). However, similar injections of DiI into retinas of p75 fl/fl; α-cre mice (n=21) reveal a TZ shifted anteriorly at P8 (FIG. 8B), as observed in p75^{-/-} mice. Quantification of the AP position of the TZ shows a significant difference between p75 fl/fl; α-cre mice compared to wild type littermates, confirming the anterior shift of TZs formed by p75 deficient nasal RGC axons (FIG. 9A). Further, the AP position of the TZ in every p75 fl/fl; α-cre case is positioned anterior to the mean TZ position for p75 fl/fl; cre-negative mice (FIG. 9B). In addition to this anterior shift of the TZ formed by p75 deficient nasal RGC axons in p75 fl/fl; α-cre mice, in a proportion of these cases the single focal DiI injection labels a dual TZ, with a TZ in the appropriate position and an ectopic TZ anterior to it (n=3; FIG. 8C).

As described above, in p75^{+/+}; α-cre; R-eGFP mice (n=8; FIG. 8D), the R-eGFP labeling pattern reveals the retinotopic map in the SC at a population level. As in p75^{-/-} mice, a significant anterior shift of the N^{d} in the SC of p75 fl/fl; α-cre; R-eGFP mice (n=12; FIGS. 8E and 8F) were found. In addition, numerous holes are evident in the eGFP labeling pattern in the T^{d} of the SC, in contrast to the more uniform labeling in wild type mice, indicative of aberrant anterior terminations of RGC axons from the eGFP-negative C^{d}. To quantify these mapping changes, the relative mean areas occupied by each of the three retinal projection domains in the SC was measured. Outlines of the projection domain borders for two representative wild type cases and a p75 fl/fl; α-cre; R-eGFP case demonstrate the anterior shift of the N^{d} as well as the variability in the overall projection for p75 mutant cases (FIG. 9C). Overall, compared to wild type (i.e. p75^{+/+}; α-cre; R-eGFP; n=8), the N^{d}, shows a significant increase in area in the p75 fl/fl; α-cre; R-eGFP mice (n=12) and the C^{d}, which retains p75 and therefore p75-ephrin-A reverse signaling, shows a concomitant statistically significant decrease in area (FIG. 9D). Because cre expression in the α-cre line is limited to nasal and temporal retina and is not evident elsewhere in the retinocollicular pathway (Marquardt et al., 2001; Baumer et al., 2002), the mapping phenotypes in the conditional and constitutive p75 knockout mice are due to the loss of p75 from RGC axons.

### REFERENCES

Alsina, B., Vu, T., and Cohen-Cory, S. (2001). Visualizing synapse formation in arborizing optic axons in vivo: dynamics and modulation by BDNF. Nature Neurosci. 4, 1093-1101.
Barker, P. A. (2004). p75NTR is positively promiscuous: novel partners and new insights. Neuron 42, 529-533.
Baumer, N., Marquardt, T., Stoykova, A., Ashery-Padan, R., Chowdhury, K., and Gruss P. (2002). Pax6 is required for establishing naso-temporal and dorsal characteristics of the optic vesicle. Development 129, 4535-4545.
Ben-Zvi, A., Ben-Gigi, L., Klein, H., and Behar, 0. (2007). Modulation of Semaphorin3A activity by p75 neurotrophin receptor influences peripheral axon patterning. J. Neurosci. 27, 13000-13011.
Brown, A., Yates, P. A., Burrola, P., Ortuño, D., Vaidya, A., Jessell, T. M., Pfaff, S. L., O'Leary, D. D. M., and Lemke, G. (2000). Topographic mapping from the retina to the midbrain is controlled by relative but not absolute levels of EphA receptor signaling. Cell 7, 77-88.
Carson, C., Saleh, M., Fung, F. W., Nicholson, D. W., and Roskams, A. J. (2005). Axonal dynactin p150 Glued transports caspase-8 to drive retrograde olfactory receptor neuron apoptosis. J. Neurosci. 25, 6092-6104.
Chao, M. V. (2003). Neurotrophins and their receptors: a convergence point for many signaling pathways. Nat. Rev. Neurosci. 4, 299-309.
Cohen-Cory, S, and Fraser, S. E. (1995). Effects of brain-derived neurotrophic factor on optic axon branching and remodelling in vivo. Nature 378, 192-196.
Cowan, C. A., and Henkemeyer, M. (2002). Ephrins in reverse, park and drive. Trends Cell Biol. 12, 339-46.
Cutforth, T., Moring, L., Mendelsohn, M., Nemes, A., Shah, N. M., Kim, M. M., Frisen, J. and Axel, R. (2003). Axonal ephrin-As and odorant receptors. Coordinate determination of the olfactory sensory map. Cell 114, 311-322.
Davy, A., Gale, N. W., Murray, E. W., Klinghoffer, R. A., Soriano, P., Feuerstein, C., and Robbins, S. M. (1999). Compartmentalized signaling by GPI-anchored ephrin-A5 requires the Fyn tyrosine kinase to regulate cellular adhesion. Genes Dev. 13, 3125-3135.
Davy, A., and Robbins, S. M. (2000). Ephrin-A5 modulates cell adhesion and morphology in an integrin-dependent manner. EMBO J. 19, 5396-5405.
Domeniconi, M., Hempstead, B. L., and Chao, M. V. (2007). Pro-NGF secreted by astrocytes promotes motor neuron cell death. Mol. Cell. Neurosci. 734, 271-279.
Feldheim, D. A., Vanderhaeghen, P., Hansen, M. J., Frisén, J., Lu, Q., Barbacid, M., and Flanagan, J. G. (1998). Topographic guidance labels in a sensory projection to the forebrain. Neuron 21, 1303-1313.
Feldheim, D. A., Kim, Y. I., Bergemann, A. D., Frisén, J., Barbacid, M., and Flanagan, J. G. (2000). Genetic analysis of ephrin-A2 and ephrin-A5 shows their requirement in multiple embodiments of retinocollicular mapping. Neuron 25, 563-574.
Flanagan, J. G. (2006). Neural map specification by gradients. Curr. Opin. Neurobiol. 16, 59-66.
Frisen, J., Yates, P. A., McLaughlin, T., Friedman, G. C., O'Leary, D. D. M., and Barbacid, M. (1998). Ephrin-A5 (AL-1/RAGS) is essential for proper retinal axon guidance and topographic mapping in the mammalian visual system. Neuron 20, 235-243.
Gale, N. W., Holland, S. J., Valenzuela, D. M., Flenniken, A., Pan, L., Ryan, T. E., Henkemeyer, M., Strebhardt, K., Hirai, H., Wilkinson, D. G., Pawson, T., Davis, S., and Yancopoulos, G. D. (1996). Eph receptors and ligands comprise two major specificity subclasses and are reciprocally compartmentalized during embryogenesis. Neuron 17, 9-19.
Grunwald., I. C. and Klein, R. (2002). Axon guidance: receptor complexes and signaling mechanisms. Current Opinion Neurobiol. 12, 250-259.
Harada, C., Harada, T., Nakamura, K., Sakai, Y., Tanaka, K., and Parada, L. F. (2006). Effect of p75NTR on the regulation of naturally occurring cell death and retinal ganglion cell number in the mouse eye. Dev. Biol. 290, 57-65.
Hattar, S., Liao, H. W., Takao, M., Berson, D. M., and Yau, K. W. (2002). Melanopsin-containing retinal ganglion cells: architecture, projections, and intrinsic photosensitivity. Science 295, 1065-1070.
Higuchi, H., Yamashita, T., Yoshikawa, H., and Tohyama, M. (2003). PKA phosphorylates the p75 receptor and regulates its localization to lipid rafts. EMBO J. 22, 1790-1800.
Hornberger, M. R., Dutting, D., Ciossek, T., Yamada, T., Handwerker, C., Lang, S., Weth, F., Huf, J., Wessel, R., Logan, C., Tanaka, H., and Drescher, U. (1999). Modulation of EphA receptor function by coexpressed ephrinA ligands on retinal ganglion cell axons. Neuron 22, 731-742.
Huai, J. and Drescher, U. (2001). An ephrin-A-dependent signaling pathway controls integrin function and is linked to the tyrosine phosphorylation of a 120-kDa protein. J. Biol. Chem. 276, 6689-6694.
Huang, E. J. and Reichardt, L. F. (2003). Trk receptors: roles in neuronal signal transduction. Annu. Rev. Biochem. 72, 609-642.
Huber, A. B., Kolodkin, A. L., Ginty, D. D., and Cloutier, J. F. (2003). Signaling at the growth cone: ligand-receptor complexes and the control of axon growth and guidance. Annual Rev. Neurosci. 26, 509-563.
Jing, S., Wen, D., Yu, Y., Holst, P. L., Luo, Y., Fang, M., Tamir, R., Antonio, L., Hu, Z., Cupples, R., Louis, J. C., Hu, S., Altrock, B. W., and Fox, G. M. (1996). GDNF-induced activation of the ret protein tyrosine kinase is mediated by GDNFR-alpha, a novel receptor for GDNF. Cell 85, 1113-1124.
Knoll, B., Zarbalis, K., Wurst, W., and Drescher, U. (2001). A role for the EphA family in the topographic targeting of vomeronasal axons. Development 128, 895-906.
Kullander, K. and Klein, R. (2002). Mechanisms and functions of Eph and ephrin signalling. Nature Rev. Mol. Cell. Biol. 3, 475-486.
Lee, K. F., Li, E., Huber, L. J., Landis, S. C., Sharpe, A. H., Chao, M. V., and Jaenisch, R. (1992). Targeted mutation of the gene encoding the low affinity NGF receptor p75 leads to deficits in the peripheral sensory nervous system. Cell 69, 737-749.
Liu, G., Beggs, H., Jürgensen, C., Park, H. T., Tang, H., Gorski, J., Jones, K. R., Reichardt, L. F., Wu, J., and Rao, Y. (2004). Netrin requires focal adhesion kinase and Src family kinases for axon outgrowth and attraction. Nat. Neurosci. 7, 1222-1232.
Marquardt, T., Ashery-Padan, R., Andrejewski, N., Scardigli, R., Guillemot, F., and Gruss, P. (2001). Pax6 is required for the multipotent state of retinal progenitor cells. Cell 105, 43-55.
Marquardt, T., Shirasaki, R., Ghosh, S., Andrews, S. E., Carter, N., Hunter, T., and Pfaff, S. L. (2005). Coexpressed EphA receptors and ephrin-A ligands mediate opposing actions on growth cone navigation from distinct membrane domains. Cell 121, 127-139.
McLaughlin, T. and O'Leary, D. D. M. (2005). Molecular gradients and development of retinotopic maps. Annu. Rev. Neurosci. 28, 327-355.
Meriane, M., Tcherkezian, J., Webber, C. A., Danek, E. I., Triki, I., McFarlane, S., Bloch-Gallego, E., and Lamarche-Vane, N. (2004). Phosphorylation of DCC by Fyn mediates Netrin-1 signaling in growth cone guidance. J. Cell Biol. 167, 687-698.
Murai, K. K. and Pasquale, E. B. (2003). ‘Eph’ ective signaling: forward, reverse and crosstalk. Journal Cell Sci. 15, 2823-2832.
Park, J. B., Yiu, G., Kaneko, S., Wang, J., Chang, J., He, X. L., Garcia, K. C., and He, Z. (2005). A TNF receptor family member, TROY, is a coreceptor with Nogo receptor in mediating the inhibitory activity of myelin inhibitors. Neuron 45, 345-351.
Parton, R. G. (1994). Ultrastructural Localization of Gangliosides; GM1 Is Concentrated in Caveolae. Journal Histochemistry and Cytochemistry 42, 155-166.
Peles, E., Nativ, M., Lustig, M., Grumet, M., Schilling, J., Martinez, R., Plowman, G. D., and Schlessinger, J. (1997). Identification of a novel contactin-associated transmembrane receptor with multiple domains implicated in protein-protein interactions. EMBO J. 16, 978-988.
Pfeiffenberger, C., Yamada, J., and Feldheim, D. A. (2006). Ephrin-As and patterned retinal activity act together in the development of topographic maps in the primary visual system. J. Neurosci. 26, 12873-12884.
Rashid, T., Upton, A. L., Blentic, A., Ciossek, T., Knoll, B., Thompson, I. D., and Drescher, U. (2005). Opposing gradients of ephrin-As and EphA7 in the superior colliculus are essential for topographic mapping in the mammalian visual system. Neuron 47, 57-69.
Rohrer, B., LaVail, M. M., Jones, K. R., and Reichardt, L. F. (2001). Neurotrophin receptor TrkB activation is not required for the postnatal survival of retinal ganglion cells in vivo. Exp. Neurol. 172, 81-91.
Sapir, T., Geiman, E. J., Wang, Z., Velasquez, T., Mitsui, S., Yoshihara, Y., Frank, E., Alvarez, F. J., and Goulding, M. (2004). Pax6 and engrailed 1 regulate two distinct embodiments of renshaw cell development. J. Neurosci. 24, 1255-1264.
Sasaki, Y., Cheng, C., Uchida, Y., Nakajima, O., Ohshima, T., Yagi, T., Taniguchi, M, Nakayama, T., Kishida, R., Kudo, Y., Ohno, S., Nakamura, F., and Goshima, Y. (2002). Fyn and Cdk5 mediate semaphorin-3A signaling, which is involved in regulation of dendrite orientation in cerebral cortex. Neuron 35, 907-920.
Sholl, D. A. (1953). Dendritic organization in the neurons of the visual and motor cortices of the cat, J. Anatomy 87, 387-406.
Simons, K. and Toomre, D. (2000). Lipid rafts and signal transduction. Nat. Rev. Mol. Cell. Biol. 1, 31-39.
Slaughter, N., Laux, I., Tu, X., Whitelegge, J., Zhu, X., Effros, R., Bickel, P., and Nela, A. (2003). The flotillins are integral membrane proteins in lipid rafts that contain TCR-associated signaling components: implications for T-cell activation. Clinical Immunology 108, 138-151.
Tessier-Lavigne, M. and Goodman, C. S. (1996). The molecular biology of axon guidance. Science 274, 1123-1133.
Trupp, M., Raynoschek, C., Belluardo, N., and Ibanez, C. F. (1998). Multiple GPI-anchored receptors control GDNF-dependent and independent activation of the c-Ret receptor tyrosine kinase. Mol. Cell. Neurosci. 11, 47-63.
Walter, J., Kern-Veits, B., Huf, J., Stolze, B., and Bonhoeffer, F. (1987). Recognition of position-specific properties of tectal cell membranes by retinal axons in vitro. Development 101, 685-696
Xiang, M., Zhou, L., Peng, Y. W., Eddy, R. L., Shows, T. B., and Nathans, J. (1993). Brn-3b: a POU domain gene expressed in a subset of retinal ganglion cells. Neuron 11, 689-701.
Yates, P. A., Holub, A. D., McLaughlin, T., Sejnowski, T. J., and O'Leary, D. D. M. (2004). Computational modeling of retinotopic map development to define contributions of EphA-ephrinA gradients, axon-axon interactions, and patterned activity. J. Neurobiol. 59, 95-113.

A number of embodiments of the invention have been described. Nevertheless, it will be understood that various modifications may be made without departing from the spirit and scope of the invention. Accordingly, other embodiments are within the scope of the following claims.

What is claimed is:

1. A method of treating a neurological disease, disorder or injury, comprising:
contacting a nerve location with an antagonist agent of p75NTR.

2. A method of treating a neurological disease, disorder or injury, comprising:
contacting a nerve location with an agent that inhibits the interaction of p75NTR with an ephrin A.

3. A method of stimulating axonal outgrowth comprising contacting a nerve with an agent that inhibits the interaction of P75NTR with an ephrin A.

4. A method of stimulating axonal outgrowth comprising contacting a nerve with an agent that antagonizes p75NTR activity.

5. The method of claim 1, 2, 3, or 4, wherein the agent comprises a soluble EphA receptor extracellular domain.

6. The method of claim 1, 2, 3, or 4, wherein the agent comprises an antisense molecule that inhibits expression of p75NTR, Fyn or an ephrin A.

7. The method of claim 1, 2, 3, or 4, wherein the agent comprises an siRNA molecule that inhibits expression of a p75NTR, a Fyn or an ephrin A.

8. The method of claim 1, 2, 3, or 4, wherein the agent comprises an antibody that binds to p75NTR and inhibits the interaction of p75NTR with ephrin A.

9. The method of claim 1, 2, 3, or 4, wherein the agent comprises an antibody binds to ephrin A and inhibits the interaction of ephrin A with p75 NTR.

10. The method of claim 1, 2, 3, or 4, wherein the agent is a small molecule inhibitor.

11. The method of claim 1, 2, 3, or 4, wherein the agent is a mutant p75NTR lacking a cytoplasmic domain.

12. The method of claim 1, 2, 3, or 4, wherein the nerve location is in vivo.

13. The method of claim 1, 2, 3, or 4, wherein the agent comprises inhibits the binding of endogenous EphA to ephrin A

14. A method of treating mono or polyneuropathy comprising contacting a subject with an agent that inhibits the interaction of p75NTR with an ephrin A or an antagonist of ephrinA-p75NTR complex activity.

15. A method of treating metastasis comprising contacting a subject with a metastatic disorder or disease with an agent that promotes the interaction of p75NTR and ephrin A or with an agonist of p75-ephrin A complex activity.

16. The method of claim 15, wherein the agent comprises a polynucleotide encoding p75NTR.

17. The method of claim 15, wherein the agent comprises a polynucleotide encoding ephrin A.

18. The method of claim 15, wherein the agent comprises an agent the induces phosphorylation of Fyn.

19. The method of claim 15, wherein the agent comprises a peptidomimetic.

20. A method of screening an agent that is useful for inducing axon outgrowth or cell motility comprising contacting a cell with the agent and measuring (i) the phosphorylation of Fyn or (ii) the interaction of p75NTR and ephrin A, wherein an agent the promotes phosphorylation of Fyn or the interaction of P75NTR and ephrin A is an agent useful for simulating axon outgrowth.
